(19) United States
(12) Patent Application Publication
(10) Pub. No.: US 2024/0315701 A1
(43) Pub. Date: Sep. 26, 2024

(54) SIZER FOR LEFT ATRIAL APPENDAGE

(71) Applicant: EDWARDS LIFESCIENCES CORPORATION, Irvine, CA (US)

(72) Inventors: Manouchehr A. Miraki, Laguna Hills, CA (US); Jason Thai Le, Garden Grove, CA (US)

(21) Appl. No.: 18/679,202

(22) Filed: May 30, 2024

Related U.S. Application Data
(63) Continuation of application No. PCT/US2022/051584, filed on Dec. 1, 2022.
(60) Provisional application No. 63/265,017, filed on Dec. 6, 2021.

(51) Int. Cl. A61B 17/12

(52) U.S. Cl. CPC A61B 17/12122; A61B 17/12159

(57) ABSTRACT

A sizer may be configured to determine a size of a portion of a heart. The portion of the heart may comprise a left atrial appendage (LAA). The sizer may include an anchor configured to anchor to the heart, and one or more indicators on the elongate body and configured to indicate a size of the portion of the heart.

### CROSS-REFERENCE TO RELATED APPLICATIONS

This application is a continuation of International Patent Application No. PCT/US2022/051584, filed Dec. 1, 2022, which claims the benefit of U.S. Patent Application No. 63/265,017, filed Dec. 6, 2021, the entire disclosures all of which are incorporated by reference for all purposes.

### BACKGROUND

### Field

Various examples disclosed herein relate generally to sizers for medical implementation. Some examples relate to sizers and clips for a left atrial appendage (LAA).

### Background

Cardiac Arrhythmias are abnormal heart rhythms that can cause the heart to pump blood less effectively. Atrial fibrillation (AF) is one of the most common heart arrhythmia conditions. AF causes the left atrium to beat irregularly and reduces the efficiency of the “atrial kick” that helps to move blood into the left ventricle.

The left atrial appendage (LAA) is a muscular pouch located high on the free wall of the left atrium. The anatomy of the LAA is such that blood has a tendency to stagnate and form clots within the LAA. As blood flow is reduced with the progression of AF, the potential for clot formation increases tremendously.

Clots formed in the LAA can embolize into the bloodstream and move into the brain, where they can become lodged and eventually lead to stroke. It may be beneficial to close or occlude the LAA, to reduce the possibility of clots or other undesired materials from passing into the left atrium and into the bloodstream.

Left atrial appendage closure (also known as LAA closure or LAAC) is a minimally invasive procedure that is used to reduce the risk of stroke that comes as a result of atrial fibrillation.

### SUMMARY

Systems, apparatuses, and methods disclosed herein may be directed to sizers for medical implementation, including sizers for a portion of a heart. The sizers may be utilized to determine a size of the portion of the heart in order to determine a size of a clip to be deployed to the portion of the heart. The clip may be configured to close the portion of the heart, to reduce blood flow therethrough as well as passage of clots or other undesired materials.

In aspects, the sizers may be utilized to determine a diameter of a left atrial appendage (LAA). The sizer may be utilized to determine a size of a LAA clip for deployment to the LAA. The clip may be configured to close the left atrial appendage (LAA). The closure of the LAA may reduce the possibility of stroke or other maladies stemming from fluid flow with the LAA. In aspects, the sizers and the clips may be positioned exterior of the LAA.

In aspects, a sizer for a portion of a heart is disclosed. The sizer may include an elongate body including an anchor configured to anchor to the heart. The sizer may include one or more indicators on the elongate body and configured to indicate a size of the portion of the heart.

In aspects, a sizer for a portion of a heart is disclosed. The sizer may include an elongate body including an end portion having a hook. The sizer may include one or more indicators on the elongate body and configured to indicate a size of the portion of the heart.

In aspects, a method is disclosed. The method may comprise anchoring an anchor of an elongate body to a heart. The method may comprise extending the elongate body along a portion of the heart. The method may comprise utilizing one or more indicators on the elongate body to determine a size of the portion of the heart.

### BRIEF DESCRIPTION OF THE DRAWINGS

FIG. 1 shows a cross sectional schematic view of a portion of a heart.

FIG. 2 illustrates a front view of a sizer.

FIG. 3 illustrates a front perspective view of the sizer shown in FIG. 2.

FIG. 4 illustrates a rear perspective view of the sizer shown in FIG. 2.

FIG. 5 illustrates a side view of a guide shaft.

FIG. 6 illustrates a perspective view of a guide shaft.

FIG. 7 illustrates a schematic view of the sizer shown in FIG. 2 utilized to determine a size of a portion of a heart.

FIG. 8 illustrates a schematic view of a clip deployed to the portion of the heart shown in FIG. 7.

FIG. 9 illustrates a front perspective view of a sizer.

FIG. 10 illustrates a front view of a sizer.

FIG. 11 illustrates a front perspective view of the sizer shown in FIG. 10.

FIG. 12 illustrates a rear perspective view of the sizer shown in FIG. 10.

FIG. 13 illustrates a top view of the sizer shown in FIG. 10.

FIG. 14 illustrates a side view of a sizer.

FIG. 15 illustrates a perspective view of a sizer.

FIG. 16 illustrates a close up perspective view of the distal end portion of the guide shaft shown in FIG. 15.

FIG. 17 illustrates a perspective view of a sizer.

FIG. 18 illustrates a side cross sectional view of the sizer shown in FIG. 17.

FIG. 19 illustrates a side view of the sizer shown in FIG. 17.

FIG. 20 illustrates a side view of a sizer.

FIG. 21 illustrates a side view of the distal end portion of the guide shaft shown in FIG. 20.

FIG. 22 illustrates a side view of the proximal end portion of the guide shaft shown in FIG. 20.

FIG. 23 illustrates a side view of a sizer.

FIG. 24 illustrates a side view of the distal end portion of the guide shaft shown in FIG. 23.

FIG. 25 illustrates a side view of the proximal end portion of the guide shaft shown in FIG. 23.

FIG. 26 illustrates a perspective view of a sizer.

FIG. 27 illustrates a perspective view of a rail shaft of the sizer shown in FIG. 26.

FIG. 28 illustrates a perspective view of a slide shaft of the sizer shown in FIG. 26.

FIG. 29 illustrates a side cross sectional view of the sizer shown in FIG. 26.

FIG. 30 illustrates a side view of the distal end portion of the guide shaft shown in FIG. 26.

FIG. 31 illustrates a perspective view of the proximal end portion of the guide shaft shown in FIG. 26.

FIG. 32 illustrates a perspective view of a mid-portion of the guide shaft shown in FIG. 26.

FIG. 33 illustrates a side view of the distal end portion of the guide shaft shown in FIG. 26.

FIG. 34 illustrates a perspective view of the proximal end portion of the guide shaft shown in FIG. 26.

FIG. 35 illustrates a perspective view of a mid-portion of the guide shaft shown in FIG. 26.

FIG. 36 illustrates a side cross sectional view of the distal end portion of the guide shaft shown in FIG. 26.

FIG. 37 illustrates a perspective view of a sizer.

FIG. 38 illustrates a perspective view of a slide shaft of the sizer shown in FIG. 37.

FIG. 39 illustrates a perspective view of a mid-portion of the guide shaft shown in FIG. 37.

FIG. 40 illustrates a perspective view of a sizer.

FIG. 41 illustrates a perspective view of a slide shaft of the sizer shown in FIG. 40.

FIG. 42 illustrates a side cross sectional view of the sizer shown in FIG. 40.

FIG. 43 illustrates a side view of the distal end portion of the guide shaft shown in FIG. 40.

FIG. 44 illustrates a side view of the proximal end portion of the guide shaft shown in FIG. 40.

FIG. 45 illustrates a side view of the distal end portion of the guide shaft shown in FIG. 40.

FIG. 46 illustrates a side view of the proximal end portion of the guide shaft shown in FIG. 40.

### DETAILED DESCRIPTION

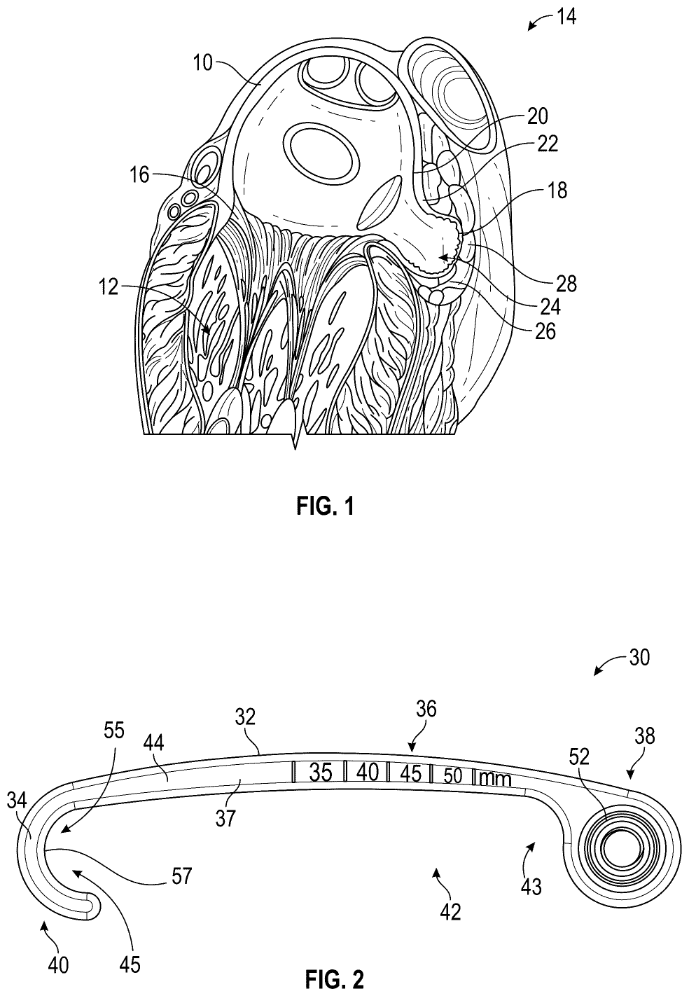

FIG. 1 illustrates a cross sectional view of a left atrium 10 and a left ventricle 12 of an individual's heart 14. The left atrium 10 is configured to fill with blood to pass into the left ventricle 12 via the mitral valve 16 during the cardiac cycle. The left atrial appendage (LAA) 18 protrudes from the outer wall 20 of the left atrium 10 and includes an ostium 22 and a cavity 24 extending from the ostium 22. A LAA wall 26 may surround the cavity 24 and may form an outer surface 28 of the LAA 18. The LAA 18 has a pouch shape extending from the left atrium 10. The cavity 24 may be configured to fill with blood, allowing the LAA to serve as a decompression chamber during systole and when pressure otherwise increases in the left atrium 10.

In certain individuals, blood may stagnate and form clots within the LAA 18. Clots or other undesired materials stemming from the LAA 18 may travel into the bloodstream, producing a variety of maladies including strokes. It may thus be beneficial to close the LAA 18, to reduce the possibility of clots or other undesired material from producing such maladies.

A clip may be utilized to close the LAA. It may be desirable to utilize a clip sized for the diameter of the LAA. As such, a sizer may be utilized to determine a diameter of the LAA to allow for an appropriately sized clip to be selected to close the LAA.

FIG. 2 illustrates an example of a sizer 30 that may be utilized according to examples herein. The sizer 30 may include an elongate body 32 that may include an anchor 34 that may be configured to anchor to the heart. One or more indicators 36 may be on the elongate body 32 and configured to indicate a size of a portion of the heart. The portion of the heart may comprise the LAA 18, or another portion of the heart. The one or more indicators 36 may be configured to indicate a diameter of the LAA 18, or another portion of the heart.

The elongate body 32 may include an arm 37, as shown in FIG. 2, which may extend from an end portion 38 of the sizer 30 to an end portion 40 of the sizer 30 that may include the anchor 34. The end portion 38 may comprise a proximal end portion and the end portion 40 may comprise a distal end portion of the elongate body 32.

The arm 37 may have a curvature, or may be straight, or may have another configuration. The arm 37 may bound a receiving area 42 for receiving a portion of the heart to be sized. For example, the arm 37 may extend along a portion of a heart (such as a left atrial appendage (LAA)), with the portion of the heart positioned within the receiving area 42. The proximal end portion 38 of the elongate body 32 may bound an end portion 43 of the receiving area 42, and the anchor 34 may bound another end portion 45 of the receiving area 42. The end portion 43 may comprise a proximal end portion of the receiving area 42 and the end portion 45 may comprise a distal end portion of the receiving area 42. The portion of the receiving area 42 opposite the arm 37 may be open to allow the portion of the heart to be passed into the receiving area 42. For example, the anchor 34 may comprise an opened hook with a gap between the tip of the hook and the proximal end portion 38 of the elongate body 32 to allow the portion of the heart to be passed through the gap and into the receiving area 42.

The receiving area 42 may have an oblong or oval shape with the length or major axis of the receiving area 42 extending along the length of the elongate body 32. The receiving area 42 may be shaped to contour to a shape of a portion of the heart to be sized (such as a left atrial appendage (LAA)). Other configurations of the receiving area 42 may be utilized.

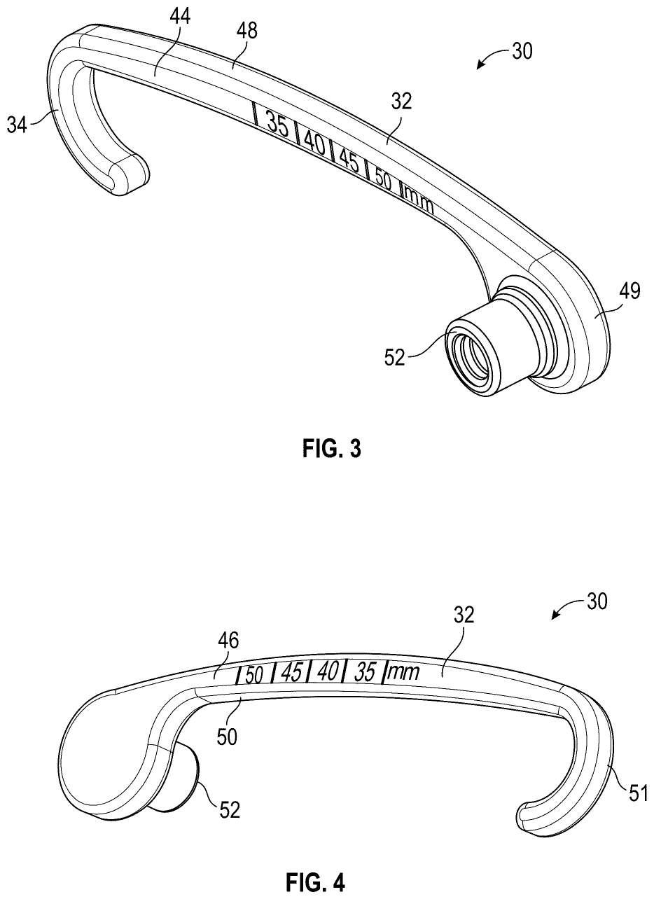

The elongate body 32 may have an oblong or oval shape, with a length being greater than a width of the elongate body 32. The elongate body 32 may have a rectangular shape or may have another shape as desired. The elongate body 32 may include a first side surface 44, a second side surface 46 (shown in FIG. 4) facing opposite the first side surface 44, a top surface 48 (shown in FIG. 3), and a bottom surface 50 (shown in FIG. 4) facing opposite the top surface 48. The elongate body 32 may include a proximal surface 49 (shown in FIG. 3) and may include a distal surface 51 (shown in FIG. 4).

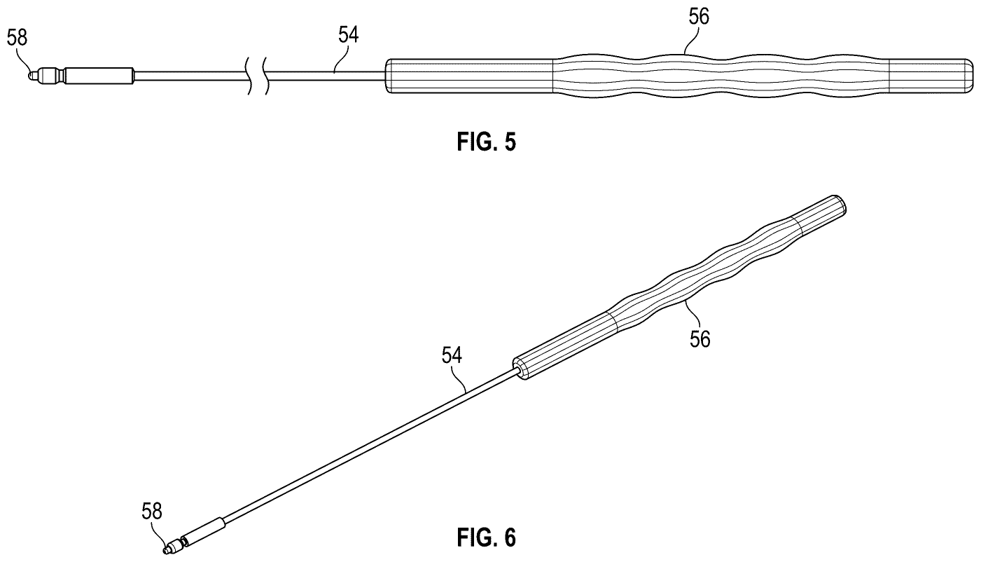

The sizer 30 may include a coupler 52 that may be configured to couple to a guide shaft 54 (shown in FIG. 5). As shown in FIG. 3, the coupler 52 may be positioned on the first side surface 44 and may be configured such that the guide shaft 54 may extend from the sizer 30 transverse or perpendicular to the plane of the elongate body 32. The length of the elongate body 32 may be angled relative to the guide shaft 54 and may extend transverse or perpendicular to the length of the guide shaft 54. The coupler 52 may include threading or may be otherwise configured to couple to the guide shaft 54.

In examples, the sizer 30 may be overmolded upon the guide shaft 54. The combination of the sizer 30 and the guide shaft 54 may be disposable. In examples, the sizer 30 may be integral with the guide shaft 54. For example, both the sizer 30 and the guide shaft 54 may be made of a material such as metal (e.g., stainless steel). The combination of the sizer and guide shaft may be reusable if desired, and sterilized prior to each use.

The one or more indicators 36 may be positioned on the elongate body 32 and may have a variety of forms. In examples, a plurality of indicators 36 may be provided that may be spaced from each other. The indicators 36 may comprise a graduated scaling upon the length of the elongate body 32. The graduations may be equally spaced or may be unevenly spaced from each other in examples. In examples, a single indicator 36 may be utilized.

The indicators 36 as shown in FIG. 2 may comprise a set of characters spaced from each other and indicating a size of a portion of a heart within the receiving area 42. The characters may comprise numbers (as shown in FIG. 2) or may have another form (e.g., letters, symbols, etc.), on the elongate body 32. The indicators 36 may further comprise markings or hash marks on the elongate body 32 that may indicate a size of a portion of a heart. The characters may indicate a size associated with an adjacent marking or hash mark. In examples, the indicators 36 may be light reflective to allow for ease of viewing during a sizing procedure.

The indicators 36 may be positioned on a side surface 44 of the elongate body 32, and may be positioned on the opposite side surface 46 (as shown in FIG. 4) or another portion of the elongate body 32 (e.g., one or more top or bottom surfaces). A combination of locations may include one or more of the indicators 36. The indicators 36 may be positioned to extend along the length of the arm 37 bounding the receiving area 42 and along the length of the receiving area 42 to allow the size of the portion of the heart within the receiving area 42 to be determined. The indicators 36 may be positioned proximate the receiving area 42 to allow for ease of comparison of the size of the portion of the heart with the one or more indicators 36.

In examples, the indicators 36 may have other forms, including surface height variations (e.g., ridges or recesses) in the surface of the elongate body 32. FIG. 10, for example, illustrates indicators 94 in the form of a graduated set of ridges upon an elongate body. The graduated set of ridges may be light reflective to allow for ease of viewing. Combinations of different forms of indicators may be utilized in examples. For example, FIG. 10 illustrates surface height variations utilized in combination with characters. Indicators may be etched or molded onto the sizer, among other forms of formation.

Features of the one or more indicators 36 may be utilized with any other example of one or more indicators disclosed herein.

Referring back to FIG. 2, the anchor 34 may have a variety of forms. The anchor 34 may comprise a hook or may have other forms (e.g., one or more barbs, clamps, clips, prongs, tethers, or other forms of anchors). FIG. 2 illustrates the anchor 34 in the form of an opened hook. The opened hook may have a curved shape and the curve may be configured to receive the portion of the heart. For example, the portion of the heart may fit within the curve 55 between the arm 37 and the tip of the hook. The curve 55 may contour to a shape of the portion of the heart. The curve 55 may bound the end portion 45 of the receiving area 42. In examples, the hook may have an angled or straightened shape.

The anchor 34 may be configured to grasp a portion of the heart to orient the indicators 36 on the elongate body 32 to the portion of the heart such the indicators 36 represent a distance from the anchor 34. The anchor 34 may comprise a datum body or zero point for the indicators 36 that the indicators 36 indicate a distance from. The anchor 34 may be configured to extend over an end of the portion of the heart and hook over that portion to position the elongate body 32 along the portion of the heart. The portion of the heart may be positioned within the receiving area 42 with the anchor 34 engaging an end of the portion of the heart. A curve 55 of the anchor 34 or other portion of the anchor 34 may comprise a datum point or portion 57 or zero point of the anchor 34 that the indicators 36 indicate a distance from. The elongate body 32 may extend along and bound a side of the receiving area 42 and the portion of the heart within the receiving area 42 such that the indicators 36 are positioned to indicate a distance from the datum point or portion 57.

The anchor, in examples, may have other configurations. For example, a closed hook shape, as shown in FIG. 10 may be utilized. Further, the anchor 34 may have other configurations (e.g., one or more barbs, clamps, clips, prongs, tethers, or other forms of anchors).

FIG. 3 illustrates a perspective view of the sizer 30. FIG. 4 illustrates a rear perspective view of the sizer 30.

In examples, the elongate body 32 may have a curvature that may be varied based on the shape of the portion of the heart to be sized. The material of the sizer 30 may be semi-soft and flexible in examples, or may be rigid as desired. The sizer may be molded with material such as polysulfone or polyetherimide (PEI, e.g., Ultum™ PEI, Sabic, Riyadh, Saudi Arabia) or it may be machined or molded out of metal such as stainless steel. Plastic, metal, or other forms of material may be utilized. The sizer may be coated with material, such as silicone to produce an atraumatic surface, or may be coated with another form of material.

FIG. 5 illustrates a side view of a guide shaft 54 that may be utilized. The guide shaft 54 may include a proximal portion with a handle 56 and may include a distal portion with a coupler 58 for coupling to the coupler 52 of the sizer 30. In examples, the guide shaft 54 may be flexible to allow for bending prior to or within the patient's body as desired. For example, the guide shaft 54 may be bent into a shape prior to insertion into the patient's body to allow for anchoring with the portion of the heart.

FIG. 6 illustrates a perspective view of the guide shaft 54. Other forms of guide shafts may be utilized as desired. In examples, use of a guide shaft may be excluded and the sizer may be utilized manually.

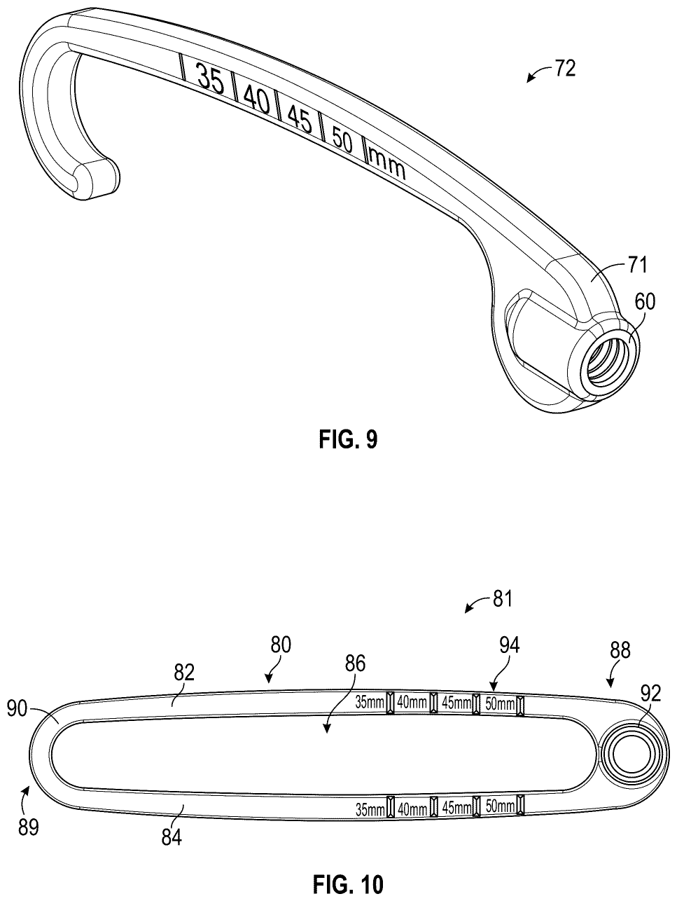

In examples, entry into the patient's body may occur in an invasive manner, such as in open chest surgery. A coupler 52 positioned as shown in FIG. 3 may be utilized to angle the elongate body 32 relative to the length of the guide shaft 54 in such an entry. In examples, entry through a port may be utilized. A configuration of a coupler 60 as shown in FIG. 9, for example, may be utilized for port entry, in which the guide shaft 54 may be aligned with the length of the sizer. Other forms of entry and configurations of couplers may be utilized.

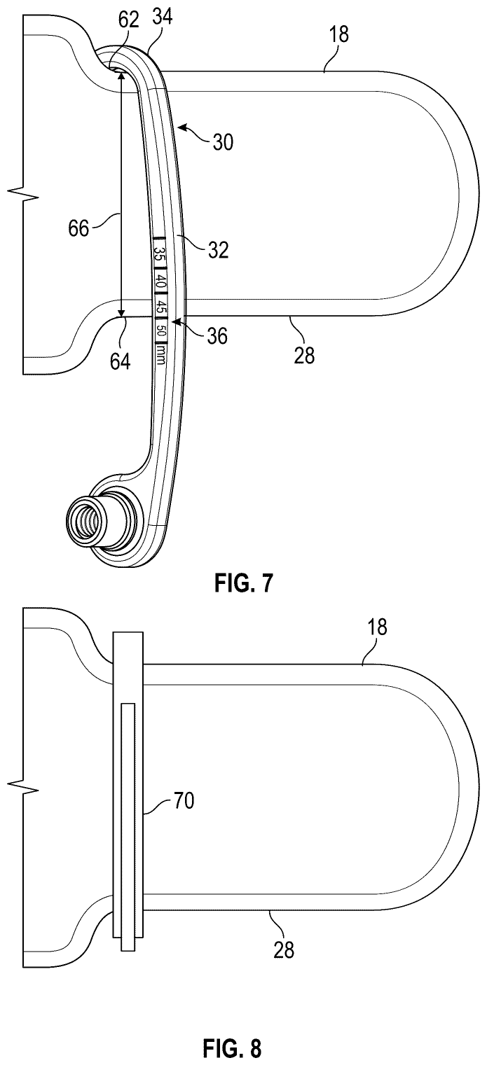

FIG. 7 illustrates an exemplary use of the sizer 30. The heart may be accessed with the heart on a bypass pump, such that blood may not be present or flowing through the heart chambers. A portion of the heart such as the left atrial appendage (LAA) 18 may thus be flattened. In examples, the heart may be accessed in a beating heart operation, with blood flowing through the heart chambers and present within a portion such as the LAA 18.

The sizer 30 may approach a portion of the heart such as the left atrial appendage (LAA) 18. The sizer 30 may be advanced towards the LAA 18 via the guide shaft 54 (not shown in FIG. 7), or in examples the use of a guide shaft 54 may be excluded.

The sizer 30 may approach perpendicular to the longitudinal axis of the LAA 18 and parallel with the diameter 66 of the LAA 18. An approach transverse to the longitudinal axis of the LAA 18 may be utilized. Other approaches may be utilized.

The anchor 34 may be anchored to the heart, which may comprise an end 62 or end wall of the LAA 18. The anchor 34 may hook around the end 62 of the LAA 18 to anchor to the LAA 18. The anchor 34 may hook around an outer surface of the LAA 18 at the base of the LAA. The base of the LAA 18 may be positioned within the curve 55 of the LAA 18 shown in FIG. 2 and may be positioned at the datum portion 57 shown in FIG. 2. The hook may serve as a tactile indicator to indicate to the user when the sizer 30 is in the desired position. For example, a user may feel the hook anchor onto the portion of the heart. Further, the hook may serve as a visual indicator to indicate to the user when the sizer 30 is in the desired position. For example, upon the tip of the hook moving out of view, the user may determine that the anchor 34 is in the desired position.

The elongate body 32 may be extended along the portion of the heart. The elongate body 32 may extend laterally along the LAA 18 at the portion of the LAA 18 to be clipped, for example, at the ostium. The one or more indicators 36 may be utilized to determine the size of the LAA 18. The indicators 36 may be positioned at the opposite end 64 of the LAA 18 and may be viewed to determine a diameter 66 of the LAA 18. The anchor 34 may be configured to anchor to a first end 62 of the portion of the heart and the one or more indicators 36 may be configured to align with a second end 64 of the portion of the heart when the anchor 34 is anchored to the heart. The diameter 66 may be measured as the distance from the datum portion 57 shown in FIG. 2 to one of the indicators 36. The size of the diameter 66 of the LAA 18, for example, may be 45 millimeters as shown in FIG. 7, and a clip sized for a 45-millimeter LAA diameter 66 may be selected.

The sizer 30 may be removed from the LAA 18. A user (e.g., a surgeon or medical practitioner) may select a clip 70 sized for the diameter 66 and may apply the clip 70 to occlude the LAA 18, as shown in FIG. 8, for example.

Features of the sizer 30 may be utilized solely or in combination with any other example disclosed herein.

In examples, other approaches and manners of measuring the diameter of the LAA may be utilized. In examples, other portions of the heart or of the patient's body may be sized utilizing the sizer 30. Variations in the configuration of the sizer 30 may be utilized.

FIG. 9, for example, illustrates an example in which a coupler 60 is positioned on a proximal surface 71 of the sizer 72. A configuration of a coupler 60 may allow the guide shaft 54 to be aligned with the length of the sizer 72. Other positions of a coupler may be utilized in examples, for example, on one or more of the side surfaces, or top or bottom surfaces. In an example, a coupler may be positioned on one of at least two side surfaces, or at another position.

Features of the sizer 72 may be utilized solely or in combination with any other example disclosed herein.

FIG. 10 illustrates an example in which the elongate body 80 of the sizer 81 includes a plurality of arms 82, 84. The arms 82, 84 may extend from the end portion 88 of the elongate body 80 to the anchor 90 positioned at the other end portion 89 of the elongate body 80. The arms 82, 84 may bound a receiving area in the form of an opening 86 for receiving a portion of the heart to be sized. An end portion 88 of the elongate body 80 may bound an end portion of the opening 86. The end portion 88 may comprise a proximal end portion of the elongate body 80 and the end portion of the opening 86 may comprise a proximal end portion. The arms 82, 84 may extend along sides of the opening 86. The opening 86 may have an oblong or oval shape as desired, or another shape, with the arms 82, 84 extending along the long sides of the opening 86. The elongate body 80 may have an oblong or oval shape, with a length greater than a width. The elongate body 80 may have a rectangular shape.

The anchor 90 may be positioned at the distal end portion 89 of the opening 86 and may close the end of the opening 86. The anchor 90 may comprise a closed hook that forms the end of the closed loop comprising the elongate body 80.

A coupler 92 may be positioned at the end portion 88 of the elongate body 80. The coupler 92 may couple to a guide shaft 54 in a similar manner as the coupler 52 shown in FIG. 3.

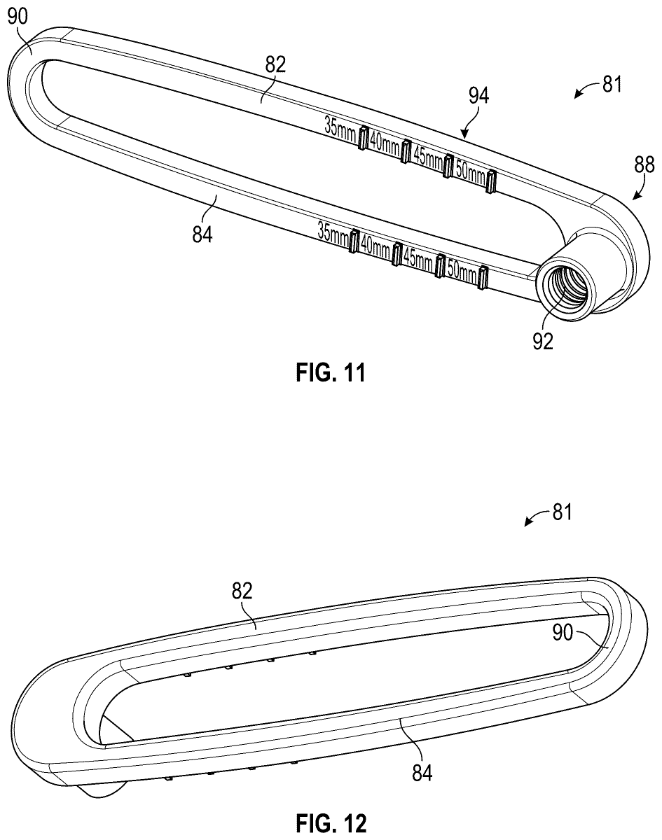

FIG. 11 illustrates a front perspective view of the sizer 81. FIG. 12 illustrates a rear perspective view of the sizer 81. The indicators 94 shown in FIGS. 10 and 11 may be positioned on a single side of the sizer 81 or may be positioned on multiple sides as desired. The indicators 94 may be raised to enhance the light reflective properties of the indicators 94.

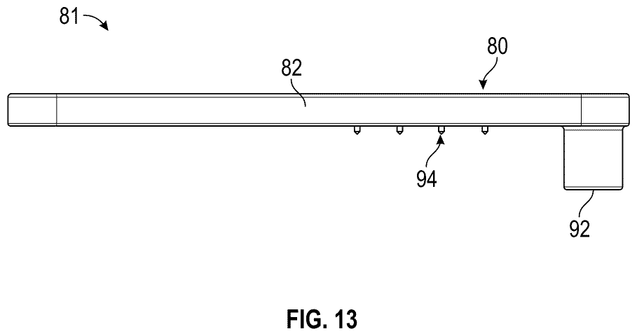

FIG. 13 illustrates a top view of the sizer 81 showing the protrusion of the indicators 94 from the surface of the elongate body 80.

In use, the sizer 81 may be slid in a direction along the longitudinal axis of the LAA, with the LAA passing through the opening 86. The LAA may pass transverse to the plane of the opening 86. The sizer 81 may be positioned at the base or ostium of the LAA and the anchor 90 may be anchored with an end of the LAA. The indicators 94 may be viewed to determine a diameter of the LAA. Other portions of a heart may be sized with the sizer 81.

Features of the sizer 81 may be utilized solely or in combination with any other example disclosed herein.

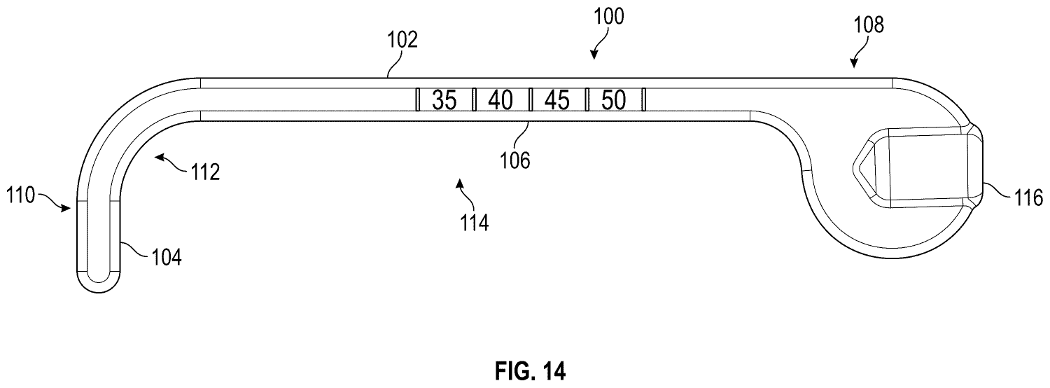

FIG. 14 illustrates a variation of the sizers shown in FIGS. 2 and 9. The sizer 100 may include features of the sizers shown in FIGS. 2 and 9 unless stated otherwise. The sizer 100 may include an elongate body 102 having an anchor 104, with at least a portion of the anchor 104 being straight. The elongate body 102 may include an arm 106 that may extend from the proximal end portion 108 of the sizer 100 to a distal end portion 110 of the sizer 100 that may include the anchor 104. The anchor 104 may extend traverse or perpendicular to the arm 106. An angled portion 112 of the elongate body 102 may connect the arm 106 to a straightened portion of the anchor 104 at the desired angle. The arm 106 may be straight. The arm 106 may have a curvature in examples.

The anchor 104 may comprise an opened hook in examples. The anchor 104 and arm 106 may bound a receiving area 114 in a similar manner as the respective anchors and arms shown in FIGS. 2 and 9. In examples, the anchor 104 may comprise a closed hook.

A coupler 116 may be configured similarly as the coupler 60 described in regard to FIG. 9. In examples, other configurations of couplers (e.g., a coupler 52 as described in regard to FIG. 2, or another form of coupler) may be utilized as desired.

The sizer 100 may operate in a similar manner as described with regard to the sizers shown in FIGS. 2 and 9.

Features of the sizer 100 may be utilized solely or in combination with any other example disclosed herein.

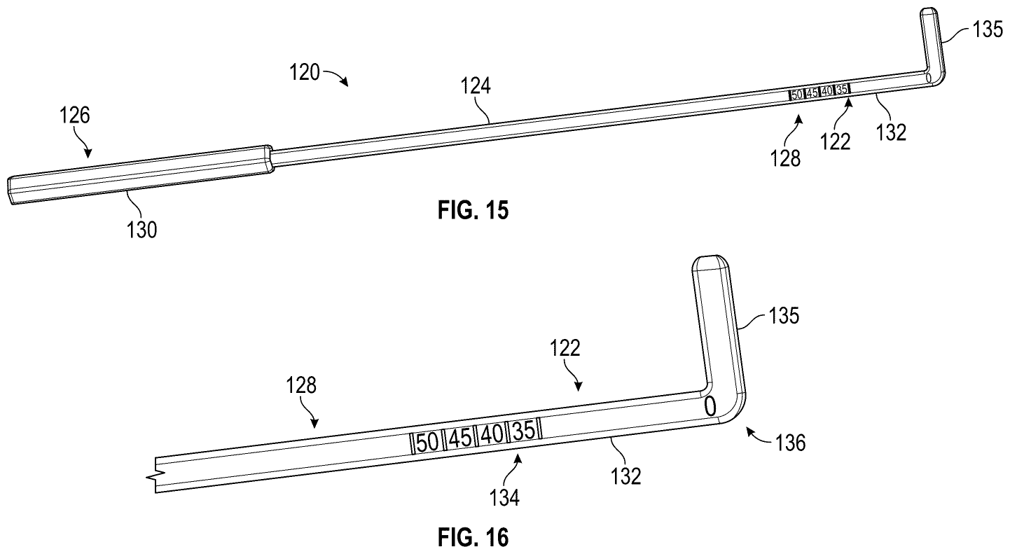

FIG. 15 illustrates an example of a sizer 120 in which the elongate body 122 is integral with the guide shaft 124. The guide shaft 124, for example, may have a first end portion or proximal end portion 126 and a second end portion or distal end portion 128. The elongate body 122 may be positioned at the distal end portion 128 of the guide shaft 124. The guide shaft 124 may be for supporting the elongate body 122, similar to other examples of guide shafts disclosed herein.

The guide shaft 124 may include a handle 130 at the proximal end portion 126 of the guide shaft 124. The handle 130 may be configured similarly as the handle 56 discussed in regard to FIGS. 5 and 6. The handle 130 may be configured for a user to grip and manipulate during a sizing procedure.

The guide shaft 124 may extend from the proximal end portion 126 to the distal end portion 128 of the guide shaft 124. The guide shaft 124 may have an elongate shape. The guide shaft 124 may comprise a metallic shaft, or may have another configuration (e.g., polymeric) as desired. The guide shaft 124 may be configured to be flexible and adjustable. The guide shaft 124 may be configured to be bent to adjust a shape of the guide shaft 124. The guide shaft 124 may configured to be bent into a desired shape for use during a sizing procedure. The guide shaft 124 may have a rectangular or square cross section, or may have another shape (e.g., rounded) as desired.

The elongate body 122 may include features of the elongate body 102 shown in FIG. 14, yet may be integral with the guide shaft 124. An arm 132 of the elongate body 122, for example, may be continuous with the guide shaft 124 and may comprise the material of the guide shaft 124. The arm 132 may have a same diameter as the guide shaft 124 or may have a different diameter or different configuration as desired. The one or more indicators 134 may be positioned on the arm 132.

FIG. 16 illustrates a close up view of the distal end portion 128 of the guide shaft 124. The anchor 135 may extend transverse or perpendicular to the arm 132, in a similar manner as the anchor 104 discussed in regard to FIG. 14. At least a portion of the anchor 135 is straight. An angled portion 136 may connect the arm 132 to the anchor 135 and may position the anchor 135 at a desired angle with respect to the arm 132. The anchor 135 may be curved in examples. The anchor 135 may comprise an opened hook. In examples, the anchor 135 may comprise a closed hook. The arm 132 may be straightened or curved in examples.

The sizer 120 may operate in a similar manner as described with regard to the sizers shown in FIGS. 2, 9, and 14.

Features of the sizer 120 may be utilized solely or in combination with any other example disclosed herein.

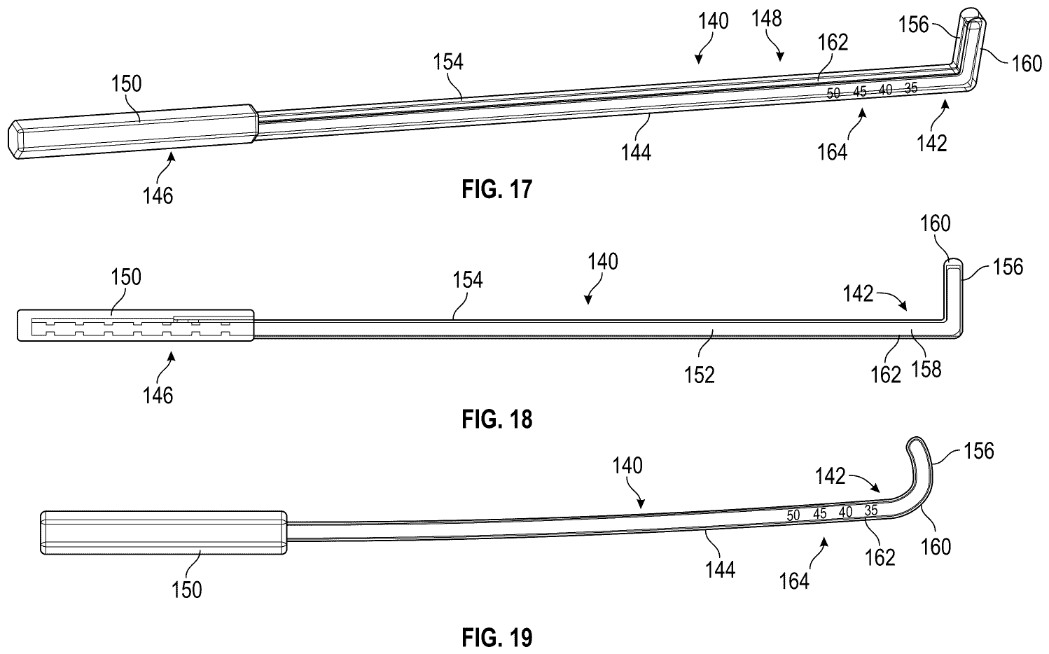

FIG. 17 illustrates a variation of the sizer 120 shown in FIG. 15. The sizer 140 may include an elongate body 142 that is integral with the guide shaft 144. The guide shaft 144 may have a first end portion or proximal end portion 146 and a second end portion or distal end portion 148. The elongate body 142 may be positioned at the distal end portion 148 of the guide shaft 144. The guide shaft 144 may be for supporting the elongate body 142, similar to other examples of guide shafts disclosed herein.

The guide shaft 144 may include a handle 150 at the proximal end portion 146 of the guide shaft 144, which may be configured similarly as the handles described in regard to FIGS. 5, 6, and 15. The handle 150 may be configured for a user to grip and manipulate during a sizing procedure.

The guide shaft 144 may extend from the proximal end portion 146 to the distal end portion 148 of the guide shaft 144. The guide shaft 144 may include a multi-body composition. For example, the guide shaft 144 may include a support shaft 152 (marked in FIG. 18) that may be positioned within a tube 154. The tube 154 may provide a variety of benefits. For example, the tube 154 may be configured to be atraumatic to an individual's body (e.g., the heart) and may reduce the possibility of damage to the body based on contact with the outer surface of the sizer 140 during a sizing procedure. In examples, the guide shaft 144 may be flexible and adjustable. The guide shaft 144 may be configured to be bent. The guide shaft 144 may be configured to be bent to adjust a shape of the guide shaft 144. The tube 154 may be flexible and may be configured to increase the strength of the support shaft 152 from breakage when the guide shaft 144 is bent. For example, the tube 154 may be durable and may comprise a strain relief for the support shaft 152.

In examples, the support shaft 152 may be metallic. The tube 154 may be elastomeric in examples. Other combinations of materials may be utilized as desired.

The elongate body 142 including the anchor 156 may have a similar composition as the guide shaft 144. The elongate body 142 and anchor 156, for example, may include a support shaft 158 (marked in FIG. 18) positioned within a tube 160. The support shaft 158 and tube 160 may be continuous with the respective support shaft 152 and tube 154 of the guide shaft 144 in examples.

At least a portion of the anchor 156 may be straight. The anchor 156 may extend perpendicular to the arm 162 in examples. The elongate body 142 may be flexible to allow the angle of the anchor 156 and the arm 162 to be varied as desired.

FIG. 18 illustrates a cross sectional view of the sizer 140 shown in FIG. 17.

The one or more indicators 164 may be positioned in a variety of locations. The one or more indicators 164 may be positioned on outer surface of the tube 160 in examples, as shown in FIG. 17.

In examples, the one or more indicators may be positioned on the support shaft 158 of the elongate body 142 in examples. The tube 160, for example, may be light transmissive to allow a user to see the one or more indicators through the tube 160 (e.g., a clear tube or transparent tube). Other locations of indicators may be utilized as desired.

The sizer 140 may operate in a similar manner as described with regard to the sizers shown in FIGS. 2, 9, 14, and 15. The guide shaft 144 may be configured to be bent to adjust a shape of the guide shaft 144. The guide shaft 144 may be bent into a desired position for a sizing procedure. The guide shaft 144 may be bent based on an approach to the portion of the heart to be sized or based on a configuration of the anatomy to be sized. FIG. 19, for example, illustrates the guide shaft 144 bent into a curve to address an approach to the portion of the heart to be sized.

In examples, the elongate body 142 may be bent as desired. For example, the angle of the anchor 156 relative to the arm 162 may be varied as desired. FIG. 19 illustrates the anchor 156 and arm 162 bent into a curve to address the native anatomy.

Features of the sizer 140 may be utilized solely or in combination with any other example disclosed herein.

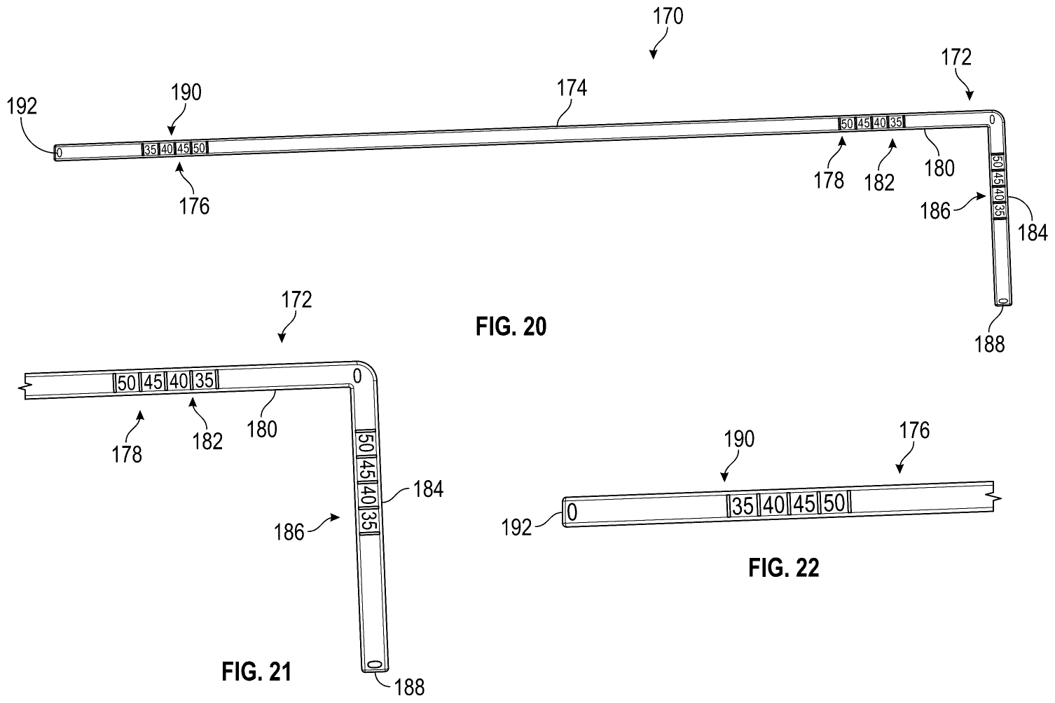

FIG. 20 illustrates a sizer 170 that may include additional positions of one or more indicators than the sizer 120 shown in FIG. 15 for example. In examples, additional portions of the sizer 170 may be utilized in a sizing procedure.

The sizer 170 may include a guide shaft 174 having a first end portion or proximal end portion 176 and a second end portion or distal end portion 178. The elongate body 172 may be integral with the guide shaft 174. The elongate body 172 may be positioned at the distal end portion 178 of the guide shaft 174. The guide shaft 174 may be for supporting the elongate body 172, similar to other examples of guide shafts disclosed herein.

FIG. 21 illustrates a close up view of the distal end portion 178 of the guide shaft 174. The elongate body 172 may include an arm 180 having one or more indicators 182. The second end portion or distal end portion 178 of the guide shaft 174 may include a hook 184. One or more indicators 186 may be positioned on the hook 184 of the elongate body 172. The hook 184 may extend perpendicular to the length of the arm 180 or at another angle as desired. The one or more indicators 186 of the hook 184 may be configured similarly as the one or more indicators 182 of the arm 180. A plurality of indicators may be provided with at least one of the indicators being positioned on the arm 180 and at least one of the indicators positioned on the hook 184. In examples, the one or more indicators 186 of the hook 184 may comprise a first set of indicators 186 and the one or more indicators 182 of the arm 180 may comprise a second set of indicators 182.

The one or more indicators 186 of the hook 184 may be utilized to indicate a size of a portion of a heart in a similar manner as the indicators 182 of the arm 180. The hook 184 may be positioned along a dimension of the portion of the heart to be sized and a length may be determined from a datum point 188 of the hook 184, which may comprise an end or tip of the hook 184. In examples, other portions of the sizer 170 may comprise a datum point.

The elongate body 172 accordingly may be utilized to size a portion of a heart in a dimension along the arm 180, as well as along a dimension of the hook 184.

In examples, a proximal end portion 176 of the guide shaft 174 may include one or more indicators 190. FIG. 22, for example, illustrates a close up view of the proximal end portion 176 of the guide shaft 174. The one or more indicators 190 may be configured similarly as the one or more indicators 182 of the arm 180. The one or more indicators 190 may comprise a third set of indicators 190 of the sizer 170. A length of a portion of the heart to be sized may be determined from a datum point 192 of the sizer 170, which may comprise an end or tip of the guide shaft 174. In examples, other portions of the sizer 170 may comprise a datum point.

The guide shaft 174 may be configured to be flexible and adjustable in examples. The guide shaft 174 may be configured to be bent to adjust a shape of the guide shaft 174. The guide shaft 174 may be configured to be bent into a desired configuration in examples.

The use of multiple locations of one or more indicators may increase the use of the sizer 170 for sizing a portion of a heart. For example, the one or more indicators 182 on the arm 180 may be utilized for sizing a diameter of a left atrial appendage (LAA) and the one or more indicators 186 on the hook 184 may be utilized to size a length of the LAA. Alternatively, the one or more indicators 186 on the hook 184 may be utilized for sizing a diameter of the LAA, and the one or more indicators 182 on the arm 180 may be utilized for sizing a length of the LAA. Simultaneous measurements may be made in examples. In a sizing procedure in which the hook 184 may be undesired (e.g., obstructive), then the one or more indicators 190 on the proximal end portion 176 of the sizer 170 may be utilized.

Various implementations of use for the sizer 170 may be provided.

Features of the sizer 170 may be utilized solely or in combination with any other example disclosed herein.

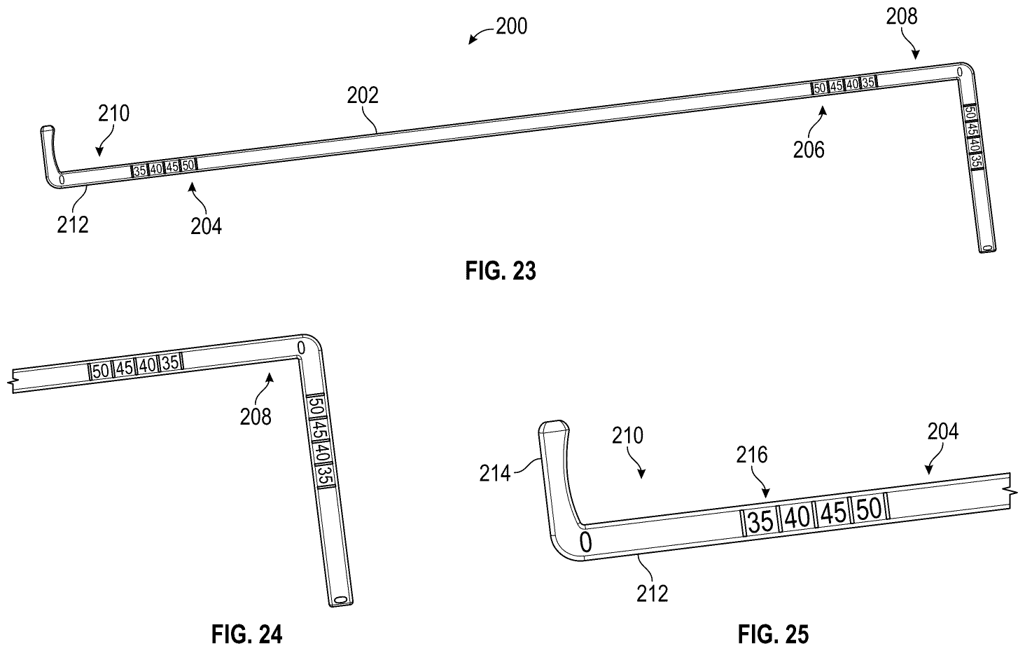

FIG. 23 illustrates a variation of the sizer 170 shown in FIG. 20. The sizer 200 may include a guide shaft 202 having a first end portion or proximal end portion 204 and a second end portion or distal end portion 206. The elongate body 208 may be integral with the guide shaft 202. The elongate body 208 may be positioned at the distal end portion 206 of the guide shaft 202. The guide shaft 202 may be for supporting the elongate body 208, similar to other examples of guide shafts disclosed herein.

The elongate body 208 may be configured similarly as the elongate body 172 discussed in regard to FIGS. 20 and 21. FIG. 24, for example, illustrates a close up view of the elongate body 208. One or more of the indicators may be positioned on the elongate body 208.

An elongate body 210, however, may be positioned at the proximal end portion 204 of the guide shaft 202. The elongate body 210 may be integral with the guide shaft 202. FIG. 25 illustrates a close up view of the proximal end portion 204 of the guide shaft 202. The elongate body 210 may include an arm 212 and an anchor 214 configured to anchor to a portion of a heart. The anchor 214 may be configured as a hook, such as an opened hook. The one or more indicators 216 on the arm 212 may be configured to size a portion of a heart in a similar manner as discussed with regard to the sizers shown in FIGS. 2, 9, 14, and 15, for example. The anchor 214 may comprise a datum point for the one or more indicators 216 on the arm 212.

The use of multiple locations of one or more indicators may increase the use of the sizer 200 for sizing a portion of a heart. For example, the plurality of indicators on the elongate body 208 may be utilized in a similar manner as discussed in regard to the sizer 170 shown in FIG. 20. The elongate body 210 at the proximal end portion 204 of the guide shaft 202 may be utilized in a sizing procedure as well. A plurality of indicators on the elongate body 210 may comprise a first set of indicators, and the plurality of indicators on the arm shown in FIG. 24 may comprise a second set of indicators. The plurality of indicators on the hook shown in FIG. 24 may comprise a third set of indicators in examples.

Features of the sizer 200 may be utilized solely or in combination with any other example disclosed herein.

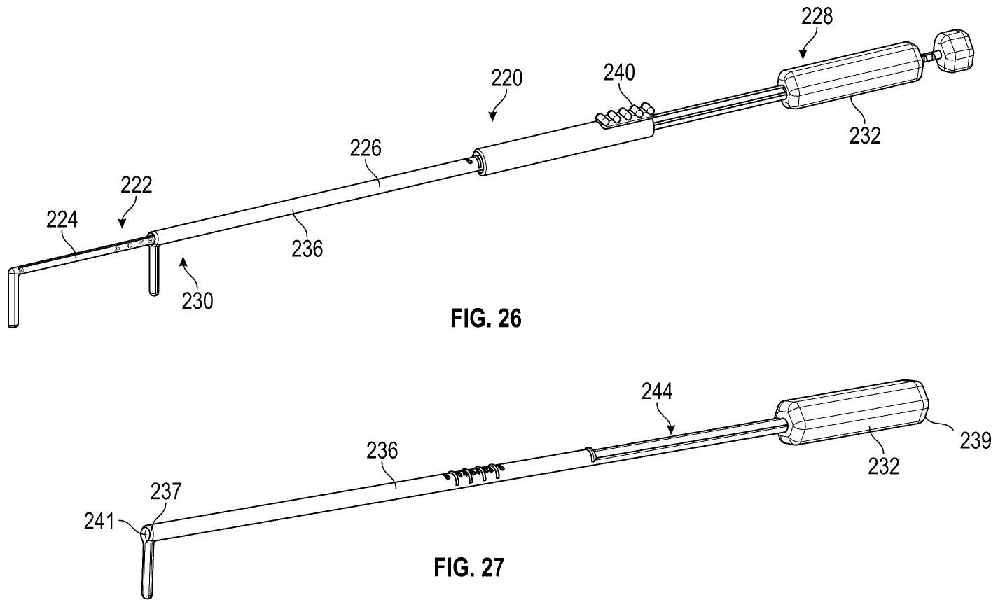

FIG. 26 illustrates a sizer 220 having an elongate body 222 with an arm 224 having an adjustable length. The sizer 220 may include a slide mechanism that may be utilized to adjust the length of the arm 224.

The sizer 220 may include a guide shaft 226 that may include a first end portion or proximal end portion 228 and a second end portion or distal end portion 230. The guide shaft 226 may be for supporting the elongate body 222, similar to other examples of guide shafts disclosed herein. The elongate body 222 may be integral with the guide shaft 226 in examples. The proximal end portion 228 of the guide shaft 226 may include a handle 232 for a user to grip. The handle 232, for example, may be configured similarly as the handles described in regard to FIGS. 5, 6, and 15. The elongate body 222 may be positioned at the distal end portion 230 of the guide shaft 226. The guide shaft 226 may comprise an elongate body in examples.

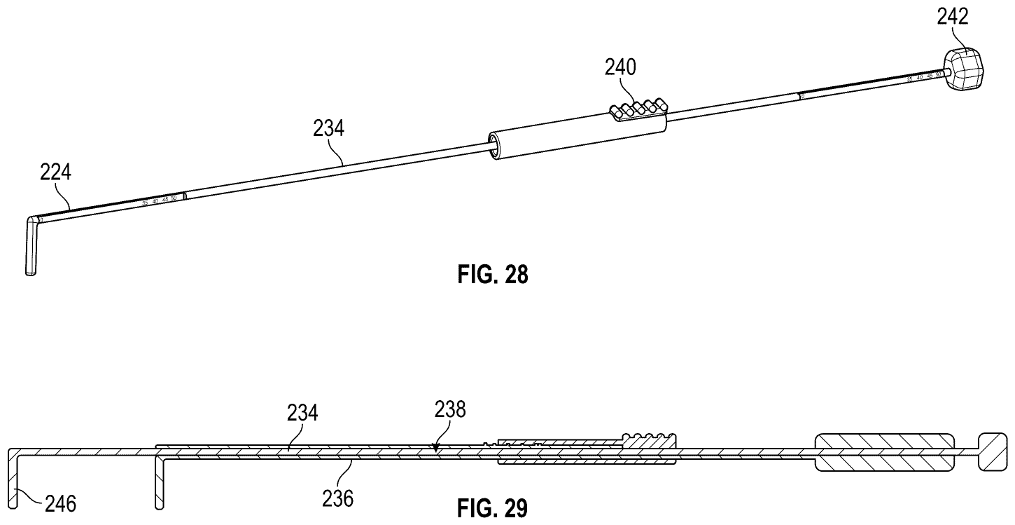

The guide shaft 226 may include a multi-body composition in examples. The guide shaft 226, for example, may include a slide shaft 234 (marked in FIG. 28) and a rail shaft 236 (marked in FIG. 27) for the slide shaft 234 to slide along. In examples, the slide shaft 234 may comprise an inner shaft and the rail shaft 236 may comprise an outer shaft or outer sheath. The rail shaft 236, for example, may include a lumen 238 (marked in FIG. 29) that the slide shaft 234 is configured to slide within.

In examples, the rail shaft 236 may include a cut out portion or channel 244 that a slide actuator 240 may slide along. The channel 244 may allow the slide actuator 240 to pass through the channel 244 and connect to the slide shaft 234.

FIG. 27 illustrates a perspective view of the rail shaft 236. The rail shaft 236 may include a distal end 237 and a proximal end 239. The distal end 237 may include an opening 241 that the arm 224 (marked in FIG. 26) may protrude from. The proximal end 239 may include an opening 243 (marked in FIG. 31) that the slide shaft 234 may protrude proximally from.

FIG. 28 illustrate a perspective view of the slide shaft 234. One or more slide actuators 240, 242 may be provided that may be utilized for adjusting the length of the arm 224. Exemplary slide actuators are shown in FIG. 28. A slide actuator 240 may be positioned on a mid-portion of the slide shaft 234 and may comprise a slide surface. The slide surface may be configured for a user to press (e.g., with a finger) to slide relative to the rail shaft 236. The movement of the slide surface may move the arm 224. A slide actuator 242 may be positioned on a proximal end portion of the slide shaft 234 and may comprise a knob. The knob may be configured for a user to grip to slide the slide shaft 234 and move the arm 224. Other forms of slide actuators 240, 242 may be utilized in examples. A single slide actuator may be utilized in examples.

FIG. 29 illustrates a side cross sectional view of the slide shaft 234 positioned within the lumen 238 of the rail shaft 236.

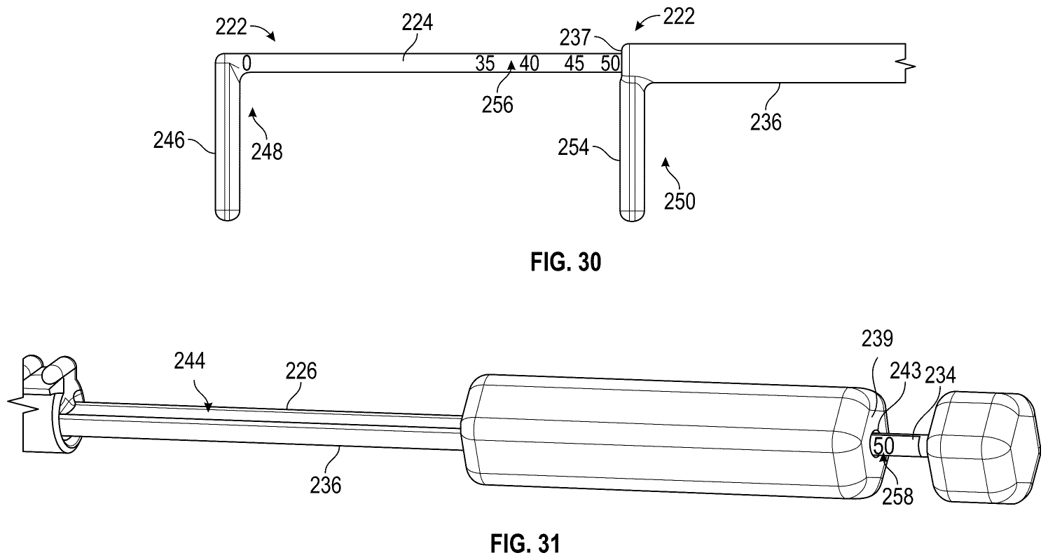

FIG. 30 illustrates a close up view of the elongate body 222. The elongate body 222 may include an anchor 246 that may be coupled to the arm 224 in a similar manner as examples of sizers disclosed herein (the sizers shown in FIGS. 2, 14, 15, and 17 for example). The anchor 246 may extend at an angle with respect to the arm 224. The anchor 246 may be positioned at a first end portion or distal end portion 248 of the elongate body 222.

A second end portion or proximal end portion 250 of the elongate body 222 may include an anchor 254. The anchor 254 may comprise a proximal anchor. At least a portion of the anchor 254 may be straight in examples. The anchor 246 may comprise a distal anchor. At least a portion of the distal anchor 246 may be straight. The anchors 254, 246 may extend perpendicular to the arm 224 in examples. The distal anchor 246 may be configured to be positioned on an opposite side of the portion of the heart to be sized (e.g., the left atrial appendage) than the proximal anchor 254.

The arm 224 may comprise a portion of the slide shaft 234. For example, the arm 224 may be integral with the slide shaft 234 in examples, and may comprise a distal end portion of the slide shaft 234.

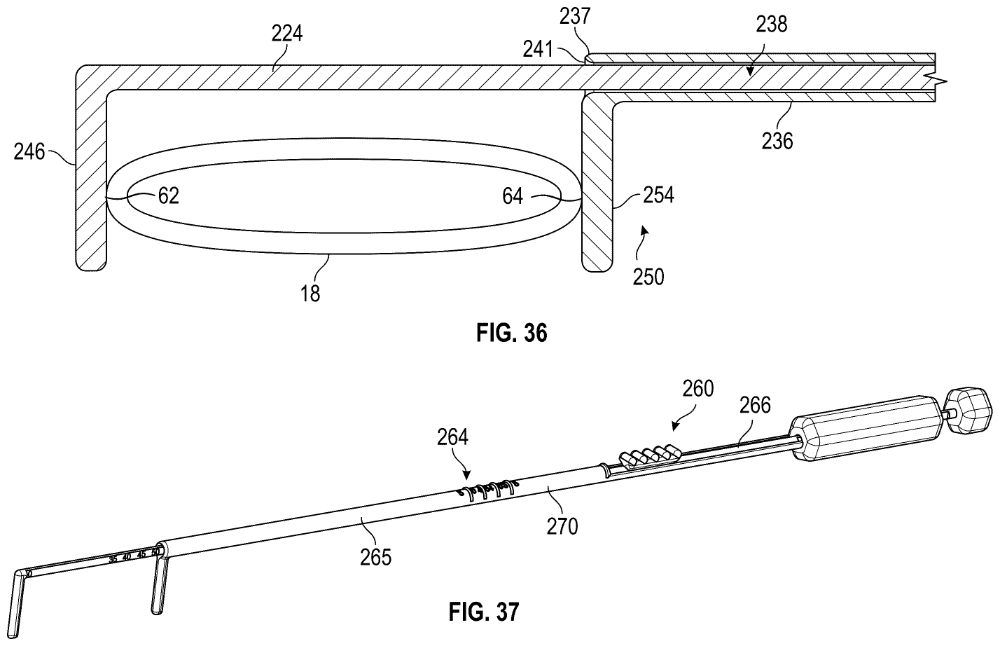

The arm 224 may be configured to protrude from the opening 241 (marked in FIG. 36) at the distal end 237 of the rail shaft 236. The length of the arm 224 that extends relative to the proximal end portion 250 of the elongate body 222 or relative to the proximal anchor 254 is adjustable and may be varied when the slide shaft 234 slides relative to the rail shaft 236. A length of the arm 224 from the second end portion or proximal end portion 250 of the elongate body 222 to the anchor 246 may be adjustable. The slide mechanism may be utilized to adjust the length of the arm 224 from the second end portion or proximal end portion 250 of the elongate body 222 to the anchor 246.

One or more indicators 256 may be positioned on the arm 224 such that the indicator displayed on the arm 224 varies in relation to the proximal end portion 250 of the elongate body 222 or relative to the proximal anchor 254. For example, a greater length of the arm 224 protruding from the proximal end portion 250 of the elongate body 252 or the proximal anchor 254 may indicate a greater size (e.g., diameter) of the portion of the heart to be sized. An indicator may be displayed that indicates this length (e.g., the 50 millimeter indicator shown in FIG. 30). The indicator may comprise the indicator shown at the distal end 237 of the rail shaft 236. A lesser length of the arm 224 extending from the proximal end portion 250 of the elongate body 222 or the proximal anchor 254 may indicate a lesser size (e.g., diameter) of the portion of the heart to be sized. An indicator may be displayed that indicates this length (e.g., a 40, 35, or 0 millimeter indicator shown in FIG. 30). An amount of the indicators 256 provided relative to a datum point (the distal end 237 of the rail shaft or the proximal anchor 254) may vary upon the length of the arm 224 being adjusted.

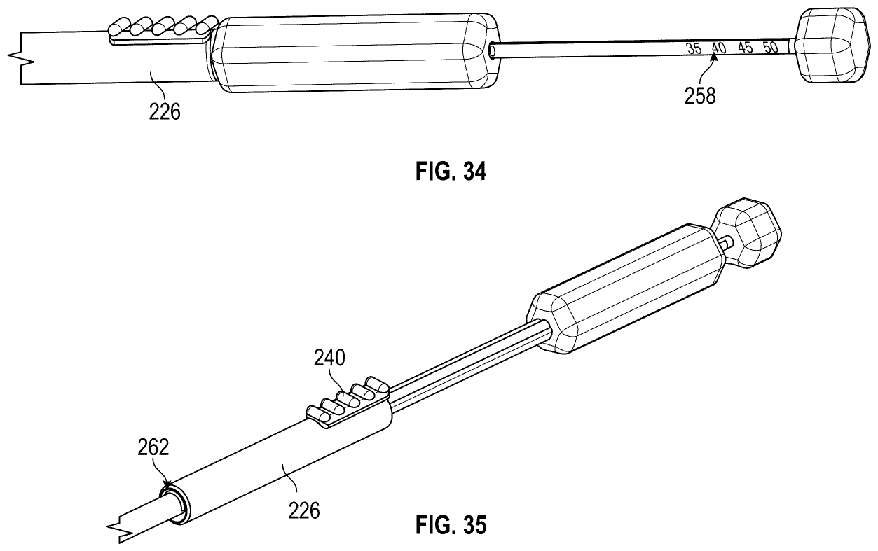

Additional indicators may be provided as desired. For example, referring to FIG. 31, one or more indicators 258 (also marked in FIG. 34) may be provided at a proximal end portion of the guide shaft 226 in examples. The one or more indicators 258 may be positioned on the slide shaft 234 for example. The indicator displayed on the slide shaft 234 may vary in relation to the proximal end 239 of the rail shaft 236. A greater protrusion of the arm 224 from the distal end 237 of the rail shaft 236 (marked in FIG. 30) may result in a lesser protrusion of the slide shaft 234 from the proximal end 239 of the rail shaft 236. Further, a lesser protrusion of the arm 224 from the distal end 237 of the rail shaft 236 may result in a greater protrusion of the slide shaft 234 from the proximal end 239 of the rail shaft 236. The indicator 258 disclosed on the proximal end of the slide shaft 234 at a datum point (the proximal end 239 of the rail shaft 236) accordingly may vary and correspond to the size (e.g., diameter) of the portion of the heart to be sized. An amount of the indicators 258 provided relative to the datum point may vary upon the length of the arm 224 being adjusted.

In examples, one or more indicators 256 may be provided solely on the arm 224, as shown in FIG. 30 for example. In examples, one or more indicators 258 may be provided solely on the proximal end of the slide shaft 234 as shown in FIGS. 31 and 34 for example. In examples, combinations of indicators at varied locations may be utilized.

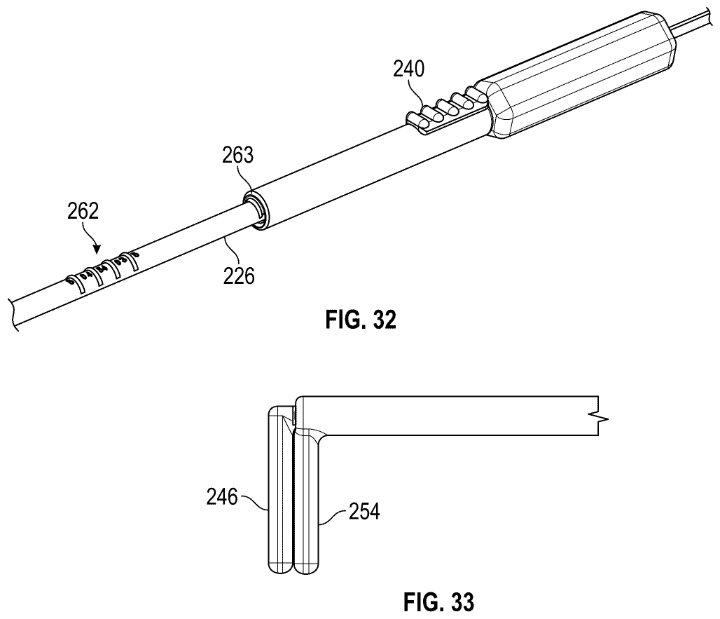

In examples, additional locations for one or more indicators may be provided. For example, referring to FIG. 32, one or more indicators 262 may be provided on a mid-portion of the guide shaft 226. A portion of the slide actuator 240 (e.g., an end 263 of the slide actuator 240) may comprise a datum point that may be configured to align with the one or more indicators 262 to indicate the size determined by the sizer 220.

The one or more indicators 262 may be utilized solely or in combination with any other indicator disclosed herein. For example, the plurality of indicators 256 on the arm 224 may comprise a first set of indicators, and the plurality of indicators 258 at the proximal end portion of the guide shaft may comprise a second set of indicators. The plurality of indicators 262 on the mid-portion of the guide shaft 226 may comprise a third set of indicators. Various other locations of indicators may be provided as desired. In examples, a single location of indicators or a single set of indicators, or single indicator may be utilized.

In operation, the guide shaft 226 may approach a portion of the heart to be sized. The arm 224 may be retracted to a position as shown in FIG. 33 for example. A zero size measurement may be shown at one or more of the indicators 256, 258, or 262. For example, referring to FIG. 34, a zero size measurement may be shown. A zero size measurement may be shown for the indicators 262 as represented in FIG. 32.

At a desired time, the length of the arm 224 may be adjusted. For example, one or more of the slide actuators 240, 242 may be utilized to extend the arm 224 relative to the proximal end portion 250 of the elongate body 222 or the proximal anchor 254 (marked in FIG. 30).

FIG. 36 illustrates a cross sectional view of the arm 224 extended with the portion of the heart (e.g., the left atrial appendage (LAA) 18) positioned between the anchors 246, 254. The arm 224 may extend across a diameter 66 of the LAA 18 in a similar manner as represented in FIG. 7. The distal anchor 246 may be positioned on one end 62 of the LAA 18 and the proximal anchor 254 may be positioned on the opposite end 64 of the LAA 18. Various other approaches may be utilized.

The one or more indicators 256 displayed on the arm 224 may accordingly vary and correspond to the measured length (e.g., 50 millimeters as shown in FIG. 30). The one or more indicators 258 shown in FIG. 31 may further display the measured length. One or more of the indicators 262 on the mid-portion of the guide shaft 226 may indicate the measured length as represented in FIG. 35.

A user may determine the length of a clip to be deployed to the portion of the heart based on the measured length.

Features of the sizer 220 may be utilized solely or in combination with any other example disclosed herein.

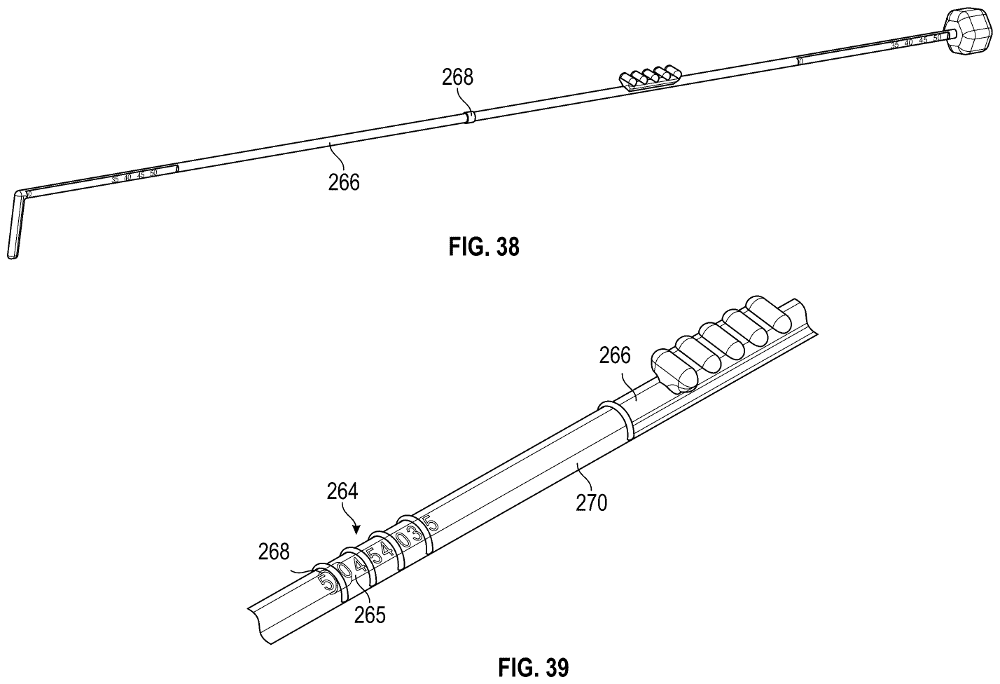

Variations in the sizer 220 may be provided in examples. For example, referring to FIG. 37, a variation of the sizer 220 including one or more indicators having a different form is shown. The sizer 260 may include features of the sizer 220 unless stated otherwise. The sizer 260 include a rail shaft 265 having one or more indicators 264 positioned thereon. Referring to FIG. 38, the slide shaft 266 of the sizer 260 may include a datum point 268 positioned thereon that may be configured to slide along with the slide shaft 266 during a sizing procedure.

Referring to FIG. 39, the rail shaft 270 may include a portion that is light transmissive to allow the datum point 268 to be visible through the rail shaft 270. The one or more indicators 264 may be static upon the movement of the rail shaft 270. The datum point 268 may slide relative to the one or more indicators 264. The datum point 268 may be aligned with one of the one or more indicators 264 to indicate the size (e.g., diameter) of the portion of the heart being sized. A user may view the alignment of the datum point 268 with the one or more indicators 264 through the rail shaft 270 or in another manner.

In examples, the one or more indicators 264 may be provided on the slide shaft 266 and a datum point 268 may be positioned on the rail shaft 270. Various configurations of indicators may be utilized.

The sizer 260 may otherwise operate in a similar manner as the sizer 220 and may include similar components as the sizer 220.

Features of the sizer 260 may be utilized solely or in combination with any other example disclosed herein.

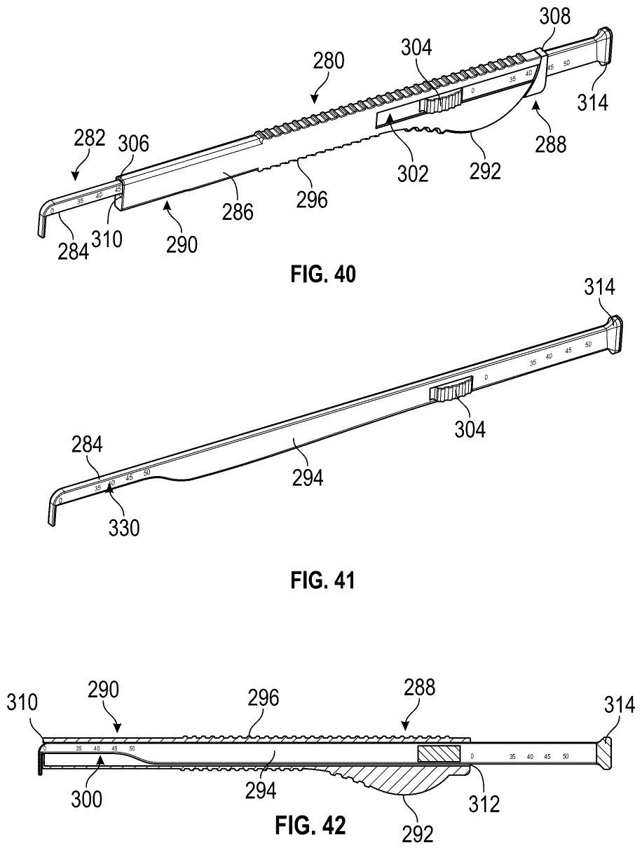

Variations in the sizers 220, 260 may be provided in examples. FIG. 40, for example, illustrates a variation. FIG. 40 illustrates a sizer 280 having an elongate body 282 with an arm 284 having an adjustable length. The sizer 280 may include a slide mechanism that may be utilized to adjust the length of the arm 284.

The sizer 280 may include a guide shaft 286 that may include a first end portion or proximal end portion 288 and a second end portion or distal end portion 290. The elongate body 282 may be integral with the guide shaft 286. The proximal end portion 288 of the guide shaft 286 may include a handle 292 for a user to grip. The elongate body 282 may be positioned at the distal end portion 290 of the guide shaft 286. The guide shaft 286 may be for supporting the elongate body 282, similar to other examples of guide shafts disclosed herein. The guide shaft 286 may comprise an elongate body in examples.

The guide shaft 286 may be configured similarly as the guide shaft 226 shown in FIG. 26. The guide shaft 286, for example, may include a multi-body composition. The guide shaft 286 may include a slide shaft 294 (marked in FIG. 41) and a rail shaft 296 for the slide shaft 234 to slide along. In examples, the slide shaft 294 may comprise an inner shaft and the rail shaft 296 may comprise an outer shaft or outer sheath. The rail shaft 296, for example, may include a lumen 300 (marked in FIG. 42) that the slide shaft 294 is configured to slide within.

In examples, the rail shaft 296 may include a cut out portion or channel 302 that a slide actuator 304 may slide along. The channel 302 may allow the slide actuator 304 to connect to the slide shaft 294 and protrude from the rail shaft 296 to be accessible by a user.

The rail shaft 296 may include a distal end 306 and a proximal end 308. The distal end 306 may include an opening 310 that the arm 284 may protrude from. The proximal end 308 may include an opening 312 (marked in FIG. 42) that the slide shaft 294 may protrude proximally from.

One or more slide actuators 304, 314 may be provided that may be utilized for adjusting the length of the arm 284. Referring to FIG. 41, for example, a slide actuator 304 may be positioned on a mid-portion of the slide shaft 294 and may comprise a slide surface. The slide surface may be configured for a user to press (e.g., with a finger) to slide relative to the rail shaft 296. The movement of the slide surface may move the arm 284. A slide actuator 314 may be positioned on an end portion of the slide shaft 294 and may comprise a knob. The knob may be configured for a user to grip to slide the slide shaft 294 and move the arm 284. Other forms of slide actuators 304, 314 may be utilized in examples. A single slide actuator may be utilized in examples.

FIG. 42 illustrates a side cross sectional view of the slide shaft 294 positioned within the lumen 300 of the rail shaft 296.

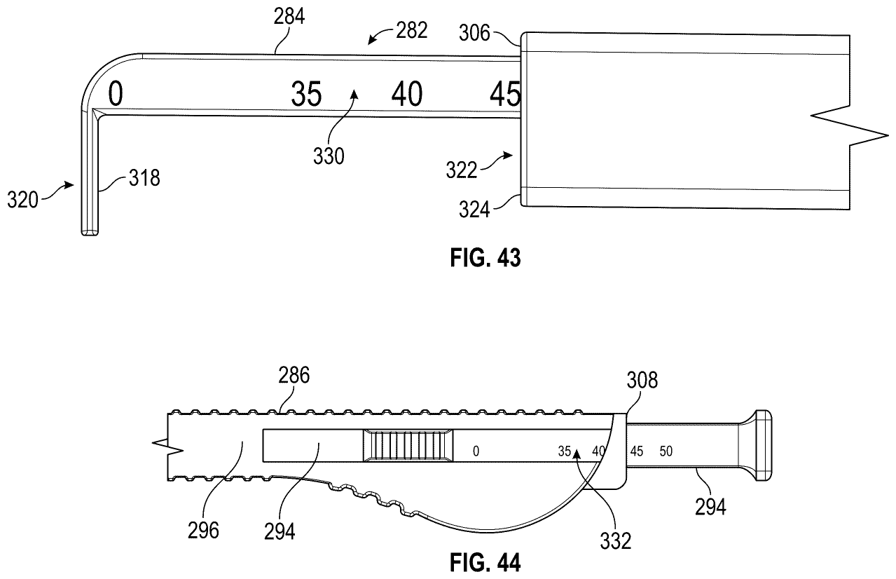

FIG. 43 illustrates a close up view of the elongate body 282. The elongate body 282 may include an anchor 318 that may be coupled to an arm 284 in a similar manner as examples of sizers disclosed herein (the sizers shown in FIGS. 2, 14, 15, and 17 for example). The anchor 318 may extend at an angle with respect to the arm 284. The anchor 318 may be positioned at a first end portion or distal end portion 320 of the elongate body 282.

A second end portion or proximal end portion 322 of the elongate body 282 may include an anchor 324. The anchor 324 may comprise a proximal anchor. The anchor 318 may comprise a distal anchor. At least a portion of the distal anchor 318 may be straight. At least a portion of the anchor 324 may be straight. The anchors 324, 318 may extend perpendicular to the arm 284 in examples. The distal anchor 318 may be configured to be positioned on an opposite side of the portion of the heart to be sized (e.g., the left atrial appendage) than the proximal anchor 324.

The arm 284 may comprise a portion of the slide shaft 294. For example, the arm 284 may be integral with the slide shaft 294 in examples, and may comprise a distal end portion of the slide shaft 294.

The arm 284 may be configured to protrude from the opening at the distal end 306 of the rail shaft 296. The length of the arm 284 that extends relative to the proximal end portion 322 of the elongate body 282 or relative to the proximal anchor 324 is adjustable and may be varied when the slide shaft 294 slides relative to the rail shaft 296. A length of the arm 284 from the second end portion or proximal end portion 322 of the elongate body 282 to the anchor 318 may be adjustable. The slide mechanism may be utilized to adjust the length of the arm 284 from the second end portion or proximal end portion 322 of the elongate body 282 to the anchor 318.

The one or more indicators 330 may be positioned on the arm 284 such that the indicator displayed on the arm 284 varies relative to the proximal end portion 322 of the elongate body 282 or relative to the proximal anchor 324. For example, a greater length of the arm 284 protruding from the proximal end portion 322 of the elongate body 282 or the proximal anchor 324 may indicate a greater size (e.g., diameter) of the portion of the heart to be sized. An indicator may be displayed that indicates this length (e.g., the 45 millimeter indicator shown in FIG. 43). An amount of the indicators 330 provided relative to a datum point (the distal end 306 of the rail shaft or the proximal anchor 324) may vary upon the length of the arm 284 being adjusted. A lesser length of the arm 284 protruding from the proximal end portion 322 of the elongate body 282 or the proximal anchor 324 may indicate a lesser size (e.g., diameter) of the portion of the heart to be sized.

Additional indicators may be provided as desired. For example, referring to FIG. 44, one or more indicators 332 may be provided at a proximal end portion of the guide shaft 286 in examples. The one or more indicators 332 may be positioned on the slide shaft 294 for example. The indicator displayed on the slide shaft 294 may vary in relation to the proximal end 308 of the rail shaft 296. A greater protrusion of the arm 284 from the distal end 306 of the rail shaft 296 may result in a lesser protrusion of the slide shaft 294 from the proximal end 308 of the rail shaft 296. Further, a lesser protrusion of the arm 284 from the distal end 306 of the rail shaft 296 may result in a greater proximal protrusion of the slide shaft 294 from the proximal end 308 of the rail shaft 296. The indicator 332 disclosed on the proximal end of the slide shaft 294 accordingly may vary and correspond to the size (e.g., diameter) of the portion of the heart to be sized. An amount of the indicators 332 provided relative to a datum point (the proximal end 308 of the rail shaft 296) may vary upon the length of the arm 284 being adjusted.

A plurality of indicators 330 on the arm 284 may comprise a first set of indicators, and the plurality of indicators 332 at the proximal end portion of the guide shaft may comprise a second set of indicators.

In examples, one or more indicators 330 may be provided solely on the arm 284, as shown in FIG. 43 for example. In examples, one or more indicators 332 may be provided solely on the proximal end of the slide shaft 294 as shown in FIG. 44 for example. In examples, combinations of indicators at varied locations may be utilized. In examples, a single location of indicators or a single set of indicators, or single indicator may be utilized.

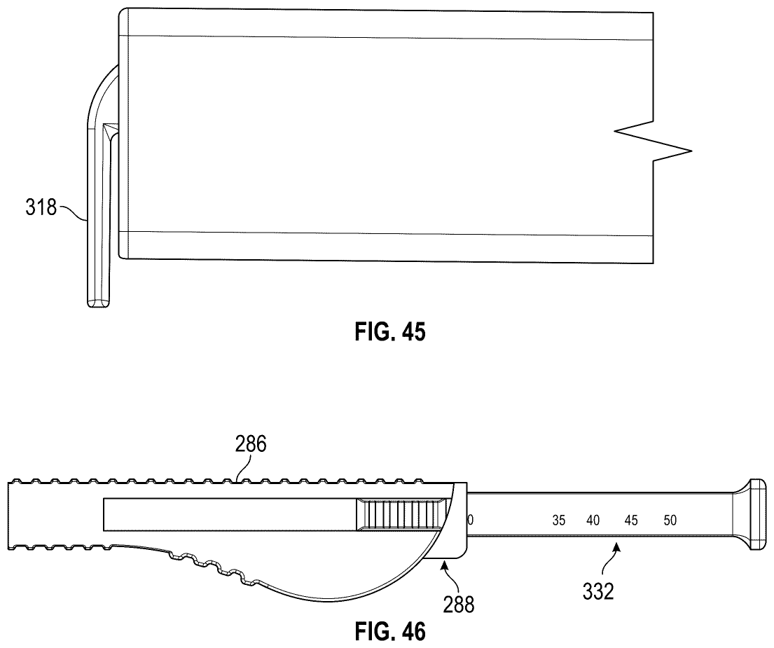

FIG. 45 illustrates the anchor 318 fully retracted. FIG. 46 illustrates the corresponding appearance of the indicators 332 at the proximal end portion 288 of the guide shaft 286.

In examples, an additional location for one or more indicators may be provided. For example, a position as shown in FIG. 32 or 39, for example, may be utilized. One or more indicators similar to indicators as shown in FIG. 32 or 39 may be utilized.

The sizer 280 may be utilized in a similar manner as the sizers shown in FIGS. 26-39 for example.

Features of the sizer 280 may be utilized solely or in combination with any other example disclosed herein.

The position of one or more indicators shown herein may not be to scale, and may be representative of a use or position of indicators.

Various other methods of deployment and use of the sizers may be provided as desired. Features across examples may be combined. Features may be excluded or added to in various examples disclosed herein. Combinations of features of examples may be provided. The sizers may be utilized solely or in methods disclosed herein, or in combination with other devices disclosed herein.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Example 1: A sizer for a portion of a heart, the sizer comprising: an elongate body including an anchor configured to anchor to the heart; and one or more indicators on the elongate body and configured to indicate a size of the portion of the heart.

Example 2: The sizer of any example herein, in particular Example 1, wherein the one or more indicators are configured to align with an end of the portion of the heart when the anchor is anchored to the heart.

Example 3: The sizer of any example herein, in particular Example 1 or Example 2, wherein the anchor is positioned at a first end portion of the elongate body, and the elongate body includes one or more arms extending from a second end portion of the elongate body to the anchor.

Example 4: The sizer of any example herein, in particular Example 3, wherein the one or more arms bound a receiving area for receiving the portion of the heart and the anchor bounds an end portion of the receiving area.

Example 5: The sizer of any example herein, in particular Example 4, wherein the receiving area comprises an opening, and the elongate body includes at least two of the arms bounding the opening.

Example 6: The sizer of any example herein, in particular Examples 1-5, wherein the anchor comprises a hook.

Example 7: The sizer of any example herein, in particular Example 6, wherein the hook comprises an opened hook or a closed hook.

Example 8: The sizer of any example herein, in particular Examples 1-7, wherein the anchor is configured to anchor to a first end of the portion of the heart, and the one or more indicators are configured to align with a second end of the portion of the heart.

Example 9: The sizer of any example herein, in particular Examples 1-8, wherein the elongate body includes a coupler for coupling to a guide shaft.

Example 10: The sizer of any example herein, in particular Example 9, wherein the elongate body includes a proximal surface, a distal surface, and at least two side surfaces extending between the proximal surface and the distal surface, and the coupler is positioned on one of the at least two side surfaces.

Example 11: The sizer of any example herein, in particular Example 9, wherein the elongate body includes a proximal surface, a distal surface, and at least two side surfaces extending between the proximal surface and the distal surface, and the coupler is positioned on the proximal surface.

Example 12: The sizer of any example herein, in particular Examples 1-11, wherein the one or more indicators include a plurality of the indicators spaced from each other.

Example 13: The sizer of any example herein, in particular Examples 1-12, wherein the one or more indicators comprise one or more of characters or surface height variations of the elongate body.

Example 14: The sizer of any example herein, in particular Examples 1-13, wherein the one or more indicators are light reflective.

Example 15: The sizer of any example herein, in particular Examples 1-14, wherein the portion of the heart comprises a left atrial appendage, and the one or more indicators are configured to indicate a diameter of the left atrial appendage.

Example 16: The sizer of any example herein, in particular Examples 1-15, wherein at least a portion of the anchor is straight.

Example 17: The sizer of any example herein, in particular Examples 1-16, wherein the elongate body includes an arm having the one or more indicators positioned thereon, and the anchor extends perpendicular to the arm.

Example 18: The sizer of any example herein, in particular Examples 1-17, further comprising a guide shaft for supporting the elongate body.

Example 19: The sizer of any example herein, in particular Example 18, wherein the one or more indicators comprise a plurality of the indicators, and wherein the guide shaft includes a first end portion and a second end portion, and the first end portion includes the elongate body, and the second end portion includes at least one of the indicators positioned thereon.

Example 20: The sizer of any example herein, in particular Example 19, wherein the elongate body includes a first set of the indicators, and the second end portion of the guide shaft includes a second set of the indicators.

Example 21: The sizer of any example herein, in particular Example 19 or Example 20, wherein the second end portion of the guide shaft includes a hook.

Example 22: The sizer of any example herein, in particular Examples 18-21, wherein the guide shaft is integral with the elongate body.

Example 23: The sizer of any example herein, in particular Examples 18-22, wherein a shape of the guide shaft is configured to be adjustable.

Example 24: The sizer of any example herein, in particular Examples 18-23, wherein the guide shaft is configured to be bent to adjust a shape of the guide shaft.

Example 25: The sizer of any example herein, in particular Examples 18-24, wherein the guide shaft includes a support shaft positioned within a tube.

Example 26: The sizer of any example herein, in particular Example 25, wherein the support shaft is metallic and the tube is elastomeric.

Example 27: The sizer of any example herein, in particular Examples 1-26, wherein the elongate body includes a support shaft positioned within a tube.

Example 28: The sizer of any example herein, in particular Examples 1-27, wherein the anchor is positioned at a first end portion of the elongate body, and the elongate body includes one or more arms extending from a second end portion of the elongate body to the anchor, and a length of the one or more arms from the second end portion to the anchor is adjustable.

Example 29: The sizer of any example herein, in particular Example 28, further comprising a slide mechanism for adjusting the length of the one or more arms from the second end portion to the anchor.

Example 30: The sizer of any example herein, in particular Example 29, wherein the slide mechanism includes a slide shaft and a slide actuator for adjusting the length of the one or more arms from the second end portion to the anchor.

Example 31: The sizer of any example herein, in particular Example 30, wherein the slide mechanism includes a rail shaft for the slide shaft to slide along.

Example 32: The sizer of any example herein, in particular Examples 28-31, wherein the anchor comprises a first anchor and the elongate body includes a second anchor at the second end portion of the elongate body, and the length of the one or more arms from the second anchor to the first anchor is adjustable.

Example 33: The sizer of any example herein, in particular Example 32, wherein the first anchor is configured to be positioned on a first side of the portion of the heart and the second anchor is configured to be positioned on an opposite side of the portion of the heart.

Example 34: The sizer of any example herein, in particular Examples 28-33, wherein the one or more indicators are positioned such that an amount of the one or more indicators provided relative to a datum point at the second end portion of the elongate body varies upon the length of the one or more arms being adjusted.

Example 35: The sizer of any example herein, in particular Examples 28-34, further comprising a guide shaft, wherein the elongate body is positioned at a first end portion of the guide shaft and a second end portion of the guide shaft includes one or more indicators positioned such that an amount of the one or more indicators at the second end portion of the guide shaft provided relative to a datum point at the second end portion of the guide shaft varies upon the length of the one or more arms being adjusted.

Example 36: A sizer for a portion of a heart, the sizer comprising: an elongate body including an end portion having a hook; and one or more indicators on the elongate body and configured to indicate a size of the portion of the heart.

Example 37: The sizer of any example herein, in particular Example 36, wherein the hook is configured to anchor to the heart.

Example 38: The sizer of any example herein, in particular Example 36 or Example 37, wherein the hook comprises an opened hook or a closed hook.

Example 39: The sizer of any example herein, in particular Examples 36-38, wherein the elongate body includes one or more arms bounding a receiving area for receiving the portion of the heart and the hook bounds an end portion of the receiving area.

Example 40: The sizer of any example herein, in particular Examples 36-39, wherein the portion of the heart comprises a left atrial appendage, and the one or more indicators are configured to indicate a diameter of the left atrial appendage.

Example 41: The sizer of any example herein, in particular Examples 36-40, wherein at least a portion of the hook is straight.

Example 42: The sizer of any example herein, in particular Examples 36-41, wherein the elongate body includes an arm having the one or more indicators positioned thereon, and the hook extends perpendicular to the arm.

Example 43: The sizer of any example herein, in particular Examples 36-42, wherein the one or more indicators are positioned on the hook.

Example 44: The sizer of any example herein, in particular Examples 36-43, wherein the one or more indicators comprise a plurality of the indicators, and the elongate body includes an arm having at least one of the indicators positioned thereon, and at least one of the indicators is positioned on the hook.

Example 45: The sizer of any example herein, in particular Examples 36-44, further comprising a guide shaft for supporting the elongate body.

Example 46: The sizer of any example herein, in particular Example 45, wherein the one or more indicators comprises a plurality of the indicators, and wherein the guide shaft includes a first end portion and a second end portion, and the first end portion includes the elongate body, and the second end portion includes at least one of the indicators positioned thereon.

Example 47: The sizer of any example herein, in particular Example 45 or Example 46, wherein the guide shaft is integral with the elongate body.

Example 48: The sizer of any example herein, in particular Examples 36-47, wherein the hook is positioned at a first end portion of the elongate body, and the elongate body includes one or more arms extending from a second end portion of the elongate body to the hook, and a length of the one or more arms from the second end portion to the hook is adjustable.

Example 49: The sizer of any example herein, in particular Example 48, further comprising a slide mechanism for adjusting the length of the one or more arms from the second end portion to the hook.

Example 50: The sizer of any example herein, in particular Example 48 or Example 49, wherein the one or more indicators are positioned such that an amount of the one or more indicators provided relative to a datum point at the second end portion of the elongate body varies upon the length of the one or more arms being adjusted.

Example 51: A method comprising: anchoring an anchor of an elongate body to a heart; extending the elongate body along a portion of the heart; and utilizing one or more indicators on the elongate body to determine a size of the portion of the heart.

Example 52: The method of any example herein, in particular Example 51, further comprising aligning the one or more indicators with an end of the portion of the heart when the anchor is anchored to the heart.

Example 53: The method of any example herein, in particular Example 51 or Example 52, wherein the anchor is positioned at a first end portion of the elongate body, and the elongate body includes one or more arms extending from a second end portion of the elongate body to the anchor.

Example 54: The method of any example herein, in particular Example 53, wherein the one or more arms bound a receiving area for receiving the portion of the heart and the anchor bounds an end portion of the receiving area.

Example 55: The method of any example herein, in particular Example 54, wherein the receiving area comprises an opening, and the elongate body includes at least two of the arms bounding the opening.

Example 56: The method of any example herein, in particular Example 54 or Example 55, wherein the anchor comprises a hook.

Example 57: The method of any example herein, in particular Example 56, wherein the hook comprises an opened hook or a closed hook.

Example 58: The method of any example herein, in particular Examples 51-57, further comprising anchoring the anchor to a first end of the portion of the heart, and aligning the one or more indicators with a second end of the portion of the heart.

Example 59: The method of any example herein, in particular Examples 51-58, wherein the elongate body includes a coupler for coupling to a guide shaft.

Example 60: The method of any example herein, in particular Example 59, wherein the elongate body includes a proximal surface, a distal surface, and at least two side surfaces extending between the proximal surface and the distal surface, and the coupler is positioned on one of the at least two side surfaces.

Example 61: The method of any example herein, in particular Example 59, wherein the elongate body includes a proximal surface, a distal surface, and at least two side surfaces extending between the proximal surface and the distal surface, and the coupler is positioned on the proximal surface.

Example 62: The method of any example herein, in particular Examples 51-61, wherein the one or more indicators include a plurality of the indicators spaced from each other.

Example 63: The method of any example herein, in particular Examples 51-62, wherein the one or more indicators comprise one or more of characters or surface height variations of the elongate body.

Example 64: The method of any example herein, in particular Examples 51-63, wherein the one or more indicators are light reflective.

Example 65: The method of any example herein, in particular Examples 51-64, wherein the portion of the heart comprises a left atrial appendage, and the one or more indicators indicate a diameter of the left atrial appendage.

Example 66: The method of any example herein, in particular Examples 51-65, wherein at least a portion of the anchor is straight.

Example 67: The method of any example herein, in particular Examples 51-66, wherein the elongate body includes an arm having the one or more indicators positioned thereon, and the anchor extends perpendicular to the arm.

Example 68: The method of any example herein, in particular Examples 51-67, further comprising a guide shaft for supporting the elongate body.

Example 69: The method of any example herein, in particular Example 68, wherein the one or more indicators comprise a plurality of the indicators, and wherein the guide shaft includes a first end portion and a second end portion, and the first end portion includes the elongate body, and the second end portion includes at least one of the indicators positioned thereon.

Example 70: The method of any example herein, in particular Example 69, wherein the elongate body includes a first set of the indicators, and the second end portion of the guide shaft includes a second set of the indicators.

Example 71: The method of any example herein, in particular Example 69 or Example 70, wherein the second end portion of the guide shaft includes a hook.

Example 72: The method of any example herein, in particular Examples 68-71, wherein the guide shaft is integral with the elongate body.

Example 73: The method of any example herein, in particular Examples 68-72, wherein a shape of the guide shaft is configured to be adjustable.

Example 74: The method of any example herein, in particular Examples 68-73, wherein the guide shaft is configured to be bent to adjust a shape of the guide shaft.

Example 75: The method of any example herein, in particular Examples 68-74, wherein the guide shaft includes a support shaft positioned within a tube.

Example 76: The method of any example herein, in particular Example 75, wherein the support shaft is metallic and the tube is elastomeric.

Example 77: The method of any example herein, in particular Examples 51-76, wherein the elongate body includes a support shaft positioned within a tube.

Example 78: The method of any example herein, in particular Examples 51-77, wherein the anchor is positioned at a first end portion of the elongate body, and the elongate body includes one or more arms extending from a second end portion of the elongate body to the anchor, and a length of the one or more arms from the second end portion to the anchor is adjustable.

Example 79: The method of any example herein, in particular Example 78, further comprising utilizing a slide mechanism to adjust the length of the one or more arms from the second end portion to the anchor.

Example 80: The method of any example herein, in particular Example 79, wherein the slide mechanism includes a slide shaft and a slide actuator for adjusting the length of the one or more arms from the second end portion to the anchor.

Example 81: The method of any example herein, in particular Example 80, wherein the slide mechanism includes a rail shaft for the slide shaft to slide along.

Example 82: The method of any example herein, in particular Examples 78-81, wherein the anchor comprises a first anchor and the elongate body includes a second anchor at the second end portion of the elongate body, and the length of the one or more arms from the second anchor to the first anchor is adjustable.

Example 83: The method of any example herein, in particular Example 82, wherein the first anchor is configured to be positioned on a first side of the portion of the heart and the second anchor is configured to be positioned on an opposite side of the portion of the heart.

Example 84: The method of any example herein, in particular Examples 78-83, wherein the one or more indicators are positioned such that an amount of the one or more indicators provided relative to a datum point at the second end portion of the elongate body varies upon the length of the one or more arms being adjusted.

Example 85: The method of any example herein, in particular Examples 78-84, further comprising a guide shaft, wherein the elongate body is positioned at a first end portion of the guide shaft and a second end portion of the guide shaft includes one or more indicators positioned such that an amount of the one or more indicators at the second end portion of the guide shaft provided relative to a datum point at the second end portion of the guide shaft varies upon the length of the one or more arms being adjusted.

Any of the features of any of the examples, including but not limited to any of the first through 85th examples referred to above, is applicable to all other aspects and examples identified herein, including but not limited to any examples of any of the first through 85th examples referred to above. Moreover, any of the features of an example of the various examples, including but not limited to any examples of any of the first through 85th examples referred to above, is independently combinable, partly or wholly with other examples described herein in any way, e.g., one, two, or three or more examples may be combinable in whole or in part. Further, any of the features of the various examples, including but not limited to any examples of any of the first through 85th examples referred to above, may be made optional to other examples. Any example of a method can be performed by a system or apparatus of another example, and any aspect or example of a system or apparatus can be configured to perform a method of another aspect or example, including but not limited to any examples of any of the first through 85th examples referred to above.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some examples have been described in connection with the accompanying drawings. The figures are drawn to scale, but such scale should not be limiting, since dimensions and proportions other than what are shown are contemplated and are within the scope of the disclosure. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

While a number of examples and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A sizer for a portion of a heart, the sizer comprising:
an elongate body including an anchor configured to anchor to the heart; and
one or more indicators on the elongate body and configured to indicate a size of the portion of the heart.

2. The sizer of claim 1, wherein the one or more indicators are configured to align with an end of the portion of the heart when the anchor is anchored to the heart.

3. The sizer of claim 1, wherein the anchor is positioned at a first end portion of the elongate body, and the elongate body includes one or more arms extending from a second end portion of the elongate body to the anchor.

4. The sizer of claim 3, wherein the one or more arms bound a receiving area for receiving the portion of the heart and the anchor bounds an end portion of the receiving area.

5. The sizer of claim 4, wherein the receiving area comprises an opening, and the elongate body includes at least two of the arms bounding the opening.

6. The sizer of claim 1, wherein the anchor comprises a hook.

7. The sizer of claim 6, wherein the hook comprises an opened hook or a closed hook.

8. The sizer of claim 1, wherein the anchor is configured to anchor to a first end of the portion of the heart, and the one or more indicators are configured to align with a second end of the portion of the heart.

9. The sizer of claim 1, wherein the elongate body includes a coupler for coupling to a guide shaft.

10. The sizer of claim 9, wherein the elongate body includes a proximal surface, a distal surface, and at least two side surfaces extending between the proximal surface and the distal surface, and the coupler is positioned on one of the at least two side surfaces.

11. The sizer of claim 9, wherein the elongate body includes a proximal surface, a distal surface, and at least two side surfaces extending between the proximal surface and the distal surface, and the coupler is positioned on the proximal surface.

12. The sizer of claim 1, wherein the one or more indicators include a plurality of the indicators spaced from each other.

13. The sizer of claim 1, wherein the one or more indicators comprise one or more of characters or surface height variations of the elongate body.

14. The sizer of claim 1, wherein the one or more indicators are light reflective.

15. The sizer of claim 1, wherein the portion of the heart comprises a left atrial appendage, and the one or more indicators are configured to indicate a diameter of the left atrial appendage.

16. The sizer of claim 1, wherein at least a portion of the anchor is straight.

17. The sizer of claim 1, wherein the elongate body includes an arm having the one or more indicators positioned thereon, and the anchor extends perpendicular to the arm.

18. The sizer of claim 1, further comprising a guide shaft for supporting the elongate body.

19. The sizer of claim 18, wherein the one or more indicators comprise a plurality of the indicators, and wherein the guide shaft includes a first end portion and a second end portion, and the first end portion includes the elongate body, and the second end portion includes at least one of the indicators positioned thereon.

20. The sizer of claim 19, wherein the elongate body includes a first set of the indicators, and the second end portion of the guide shaft includes a second set of the indicators.

21. The sizer of claim 18, wherein the guide shaft is integral with the elongate body.

22. The sizer of claim 18, wherein the guide shaft is configured to be bent to adjust a shape of the guide shaft.

23. The sizer of claim 1, wherein the anchor is positioned at a first end portion of the elongate body, and the elongate body includes one or more arms extending from a second end portion of the elongate body to the anchor, and a length of the one or more arms from the second end portion to the anchor is adjustable.

24. The sizer of claim 23, further comprising a slide mechanism for adjusting the length of the one or more arms from the second end portion to the anchor.

25. The sizer of claim 23, wherein the one or more indicators are positioned such that an amount of the one or more indicators provided relative to a datum point at the second end portion of the elongate body varies upon the length of the one or more arms being adjusted.
